(19) United States
(12) Patent Application Publication
(10) Pub. No.: US 2025/0051396 A1
(43) Pub. Date: Feb. 13, 2025

(54) MODIFIED VIRAL CAPSIDS

(71) Applicants: Tomas BJÖRKLUND, Lund (SE); Marcus DAVIDSSON, Malmö (SE)

(72) Inventors: Tomas BJÖRKLUND, Lund (SE); Marcus DAVIDSSON, Malmö (SE)

(21) Appl. No.: 18/802,552

(22) Filed: Aug. 13, 2024

Related U.S. Application Data
(63) Continuation of application No. 16/970,004, filed on Aug. 14, 2020, now abandoned.

(30) Foreign Application Priority Data
Feb. 15, 2018 (EP) 18156932.8

(51) Int. Cl. C07K 14/005; C12N 15/10; C12N 15/69; C12N 15/86

(52) U.S. Cl. CPC C07K 14/005; C12N 15/1082; C12N 15/69; C12N 15/86; C12N 2750/14122; C12N 2750/14143

(57) ABSTRACT

Provided are methods for identifying polypeptides that when displayed on a capsid confer a desired property to viral particles including such capsids, as well as methods for designing and manufacturing viral vectors and viral particles with improved properties.

### CROSS REFERENCE TO RELATED APPLICATIONS

This application is a continuation of U.S. patent application Ser. No. 16/970,004 filed Aug. 14, 2020, which is a U.S. National Stage of international application PCT/EP2019/053610 filed Feb. 14, 2019, which claims priority to European Application No: 18156932.8 filed Feb. 15, 2018, the entire contents of each of which are incorporated herein by reference.

### TECHNICAL FIELD

The present invention relates to viral vectors and particles and methods and tools for designing and manufacturing the same.

Reference to sequence listing submitted electronically The instant application contains a Sequence Listing which has been submitted electronically in .xml format and is hereby incorporated by reference in its entirety. Said .xml copy, created Jul. 3, 2024, is named “Sequence Listing B.JOR0001NA.xml” and is 181,513 bytes in size.

### BACKGROUND

Engineering of viral vectors through directed evolution has generated a plethora of potent capsids with improved tropism and function¹⁻⁹. While recent additions of Cre-recombinase restricted selection and deep sequencing for optimization has improved the accuracy and potency of this approach in recent years, it is still restricted by the serial infectivity and chimeras generated during production that requires multiple generations of screening until the real functional capsids surface²⁻⁴. Due to the randomness of the process, a very small faction of the de novo sequences code for valid in frame amino-acid substitutions, and even fewer are assembled correctly. This screening approach is also inherently unreproducible and optimizations have to be conducted post hoc. The resulting capsid variants also provide little insights into the function and what molecular targets are engaged.

A commonly used alternative approach is rational design, where systematic changes are made based on the known properties of the capsid (e.g., the removal of heparan sulfate proteoglycan binding from the AAV2 capsid) or through systematic amino-acid substitutions or display of high affinity nanobodies of the capsid surface¹⁰⁻¹⁴. While functionally more stringent, this approach provides less diversity and has more restricted functional potential.

Accordingly, there is a need for methods for designing capsids with improved properties, which are reliable, reproducible and allow great diversity.

### SUMMARY

The methods and vectors provided herein overcome the drawbacks described above. By applying a rationalised, systematic approach on selected proteins known to have or suspected to have a desired property, a library of modified viral vectors encoding modified viral particles can be expressed, where the modified viral particles comprise a modified capsid displaying a fragment of the selected proteins. Using tailored screening, fragments of said proteins which are particularly useful for conferring a desired property to a viral particle can be identified. These can be used to design modified capsids, i.e. capsids displaying one of said identified fragments, with tailored properties. As can be seen in the examples, the methods can be used for example to design viral particles with increased tropism and/or infectivity for a given cell type. The present methods are reliable, reproducible and allow great diversity. The generated capsids can be used for delivering a transgene and find applications not only in methods of treatment and gene therapy, but also in e.g. functional mapping of protein domains and drug screening.

Herein is provided a method of manufacturing a library of viral vectors, comprising the steps of:
i) selecting one or more candidate polypeptides from a group of polypeptides having or suspected of having a desired property, and retrieving the sequences of said polypeptides;
ii) providing a plurality of candidate polynucleotides, each candidate polynucleotide encoding a polypeptide fragment of one of said candidate polypeptides, such that upon transcription and translation each candidate polypeptide is represented by one or more polypeptide fragments of each candidate polypeptide;
iii) providing a plurality of barcode polynucleotides;
iv) inserting each candidate polynucleotide together with a barcode polynucleotide into a viral vector, comprising a capsid gene and a viral genome, thereby obtaining a plurality of viral vectors each comprising a single candidate polynucleotide operably linked to a barcode polynucleotide, wherein the candidate polynucleotide is inserted within the capsid gene, the capsid gene is outside the viral genome and the barcode polynucleotide is inserted within the viral genome; wherein the viral vector comprises a marker polynucleotide encoding a detectable marker;
v) amplifying the plurality of viral vectors obtained in step iv) in an amplification system, wherein each viral vector is present in a plurality of copies in the amplification system; and
a) retrieving and transferring at least a first part of the plurality of viral vectors from the amplification system of step v) in a reference system, thereby mapping each barcode polynucleotide to one candidate polynucleotide; and
b) maintaining a second part of the plurality of viral vectors in the amplification system, and optionally transferring all or part of said second part in a production system to obtain a plurality of viral particles.

Also provided is a method of designing a viral vector having a desired property, comprising steps i) to v) above and further comprising the steps of:
vi) retrieving a fraction of viral vectors from the amplification system of step v) b) above, or retrieving at least part of the viral particles from the production system of step v) b) above, and contacting a cell population with said retrieved viral vectors or viral particles;
vii) monitoring marker expression and selecting the cells wherein marker expression follows a desired pattern;
viii) identifying the barcode polynucleotides expressed in the cells selected in step vii), thereby identifying the candidate polynucleotides responsible for the desired property and the corresponding candidate polypeptides;
ix) designing a viral vector comprising a modified capsid gene, wherein the modified capsid gene comprises one of the candidate polynucleotides identified in step viii).

Also provided is a method of manufacturing a viral particle having a desired property, said method comprising steps i) to v) above and further comprising the steps of:
vi) retrieving at least part of the plurality of viral vectors from the amplification system of step v) b) above or retrieving at least part of the plurality of viral particles from the production system of step v) b) above;
vii) contacting a cell population with the retrieved viral vectors or viral particles obtained in step vi);
viii) monitoring marker expression and selecting the cells wherein marker expression follows a desired pattern;
ix) identifying the barcode polynucleotides expressed in the cells identified in step viii), thereby identifying the candidate polynucleotides responsible for the desired property and the corresponding candidate polypeptides;
x) designing a viral vector comprising a modified capsid gene, wherein the modified capsid gene comprises one of the candidate polynucleotides identified in step ix);
xi) producing the viral vector of step x) in an amplification system or in a production system, thereby obtaining the viral particle having the desired property.

Also provided is a method of delivering a transgene to a target cell, said method comprising:
a) providing a modified viral vector or a modified viral particle comprising a modified capsid and encapsulating a transgene, wherein the modified viral vector or the modified viral particle is the viral vector or the viral particle defined in step xi) above; and
b) injecting said modified viral vector or said modified viral particle into an injection site.

Also provided is a library of viral vectors, each viral vector comprising:
i) a backbone for expressing the viral vector in a host cell;
ii) a capsid gene and a candidate polynucleotide inserted therein, said candidate polynucleotide encoding a polypeptide fragment of a candidate polypeptide;
iii) a marker polynucleotide; and
iv) a barcode polynucleotide;
wherein
the candidate polypeptide is selected from a predefined group comprising one or more polypeptides having or suspected to have a desired property;
wherein upon transcription and translation each candidate polypeptide is represented by one or more polypeptide fragments in the library;
the candidate polynucleotide is inserted within the capsid gene of the viral vector so that it can be transcribed and translated to a polypeptide fragment displayed on the capsid, and is operably linked to a barcode polynucleotide inserted within the viral genome,
and the marker polynucleotide is comprised within the viral genome and the capsid gene is outside the viral genome.

Also provided is a cell or a plurality or cells comprising the library of viral vectors described herein.

Also provided is a viral vector encoding a viral particle for delivery of a transgene to a target cell, said viral vector comprising a modified capsid gene and a transgene to be delivered to the target cell;
wherein
the modified capsid gene is outside the viral genome and comprises a polynucleotide encoding a polypeptide improving delivery of the transgene and/or targeting to the target cell.

Also provided are viral particles encoded by the viral vectors described herein.

Also provided is a modified viral vector or viral particle for delivery of a transgene to a target cell, said modified viral vector or viral particle comprising a modified capsid and a transgene to be delivered to the target cell;
wherein
the modified capsid improves one or more of: delivery of the transgene to the target cell, targeting to the target cell, infectivity of the modified viral vector or modified viral particle, and/or retrograde transport of the modified viral vector or modified viral particle compared to an unmodified viral particle comprising a native capsid gene and the transgene.

Also provided is the use of a viral vector, a viral particle, a modified viral vector or a modified viral particle described herein for gene therapy.

Also provided is a viral vector, a viral particle, a modified viral vector or a modified viral particle described herein for use in a method of treatment of a disorder, such as a disorder of the nervous system.

Also provided is a method of treatment of a disorder, such as a disorder of the nervous system, in a subject in need thereof, said method comprising administration of a viral vector, a viral particle, a modified viral vector or a modified viral particle described herein to the subject.

Also provided is a method for identifying a drug having a desired effect, said method comprising the steps of:
a) Providing a candidate drug;
b) Administering the candidate drug to a cell;
c) Providing a modified viral particle comprising a modified capsid allowing delivery of the viral particle to the cell of b) and a marker polynucleotide;
d) Monitoring and comparing expression and/or localisation of the marker polypeptide in the presence and absence of the candidate drug;
thereby determining whether the candidate drug has an effect on the expression of the marker polynucleotide.

Also provided is a method for improving tropism of a viral vector or particle toward a target cell, said method comprising steps i) to v) above and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having increased expression of the marker compared to the expression from the reference viral vector or reference viral particle;
x) designing a viral vector or a viral particle with improved tropism, comprising a modified capsid gene, wherein the modified capsid gene comprises one of the candidate polynucleotides identified in step ix).

Also disclosed herein is a method of identifying one or more regions of a polypeptide conferring a desired property to a viral particle comprising a capsid modified by insertion of said polypeptide therein, said method comprising steps i) to v) above and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having an expression profile of the marker corresponding to the desired property.

### DESCRIPTION OF THE DRAWINGS

FIG. 1: Generation of highly diverse AAV capsid library for the BRAVE approach

(A) Pie-chart displaying the 131 proteins with documented affinity to synapses that were identified from the literature. They fall into three major groups, Viral derived (capsid and envelope) proteins, host derived proteins (neurotrophins and disease-related proteins e.g., Tau), Neurotoxins and Lectins. (B) 1. NCBI reference sequences of the 131 proteins were translated into amino acid sequences and computationally digested into 14aa long polypeptides with a 1aa shifting sliding window approach. The 61 314 peptides generated in total consisted of 44 708 unique polypeptides. 2. Three alternative linkers were added to the polypeptides; a single Alanine (called 14aa), a ridged linker with 5 Alanine residues (14aaA5) and a flexible linker with the aa sequence GGGGS (14aaG4S). A last group of aa peptides were similarly generated with a 22aa length and a single Alanine linker (called 22aa). 3. A total of 92 358 aa sequences were then codon optimized for expression in human cells and overhangs added to the ends to allow for directional scar-less Gibson-assembly cloning into the AAV2 Cap gene at the position 588. All resulting oligos with the total length of 170 bp were synthesized in parallel on a CustomArray oliqonucleotide array. 4. The resulting pool of oligonucleotides was assembled into a novel AAV production backbone with Cis-acting AAV2 Rep/Cap and ITR-flanking CMV-GFP. A 20 bp random molecular barcode (BC) was simultaneously inserted in the 3′ UTR of the GFP gene. 5a. Using a uni-directional Cre-recombination approach followed by emPCR-based addition of Illumina sequencing adapters, the entire pool of peptides is then sequenced using Paired-end sequencing linking the random barcodes to the respective peptides in a Look-Up table (LUT). The resulting library contained 3 934 570 unique combinations of peptide and barcode. 5b. In parallel to the LUT generation, the same plasmid library is utilized to generate replication deficient AAV viral vector preps where the peptide is displayed on the capsid surface and the barcode is packaged as part of the AAV genome. Multiple parallel screening experiments are then performed both in vitro and in vivo and after suitable selection (e.g, dissection of the targeted brain region) the mRNA is extracted and the expressed barcodes sequenced. 6. Through the combination of the sequenced barcodes and the LUT, efficacy can be mapped back to the original 131 proteins and consensus motifs can be determined using the Hammock, hidden Markov-model based clustering approach (7). (C) Two separate productions of AAV libraries were performed with a plasmid concentration of 3 copies per cell (30cpc) or a ten-fold higher concentration (30cpc). To assess which peptides would allow for correct assembly and genome packaging, we DNAse treated both batches (to remove non-packaged genomes), lysed the batches and sequenced the barcodes from the preps separately. Due to the very high diversity of the expressed barcodes, the overlap in barcodes contained was very small. However, the absolute majority of all peptides packaged in the 3cpc batch were also recovered in the 30cpc batch. (D) To assess the functional contribution of the inserted peptide we injected the 30cpc library into the forebrain of adult rats and compared the expression pattern to a AAV2-WT vector batch expressing the same transgene (GFP) at the same titer.

FIG. 2 Single-generation BRAVE screening in vitro and in vivo

(A-B) In a first proof-of-concept study we decided to utilize the BRAVE technology to screen for the re-introduction of tropism for HEK293T cells in vitro. Wild type AAV2 displays very high infectivity attributed to Heparin Sulfate (HS) proteoglycan binding (B). The AAV-MNMnull serotype disrupts this binding through the insertion of an Nhel restriction enzyme site at base 587/588 and thereby significantly reduces the HEK293T cell infectivity (B′). In the screening of the 4 million uniquely barcoded capsid variants, we found several regions from the 132 included proteins that conferred a significantly improved infectivity over the parent AAV-MNMnull capsid structure. One peptide from HSV-2 surface protein pUL44 was selected and a first novel capsid was generated named AAV-MNM001. This capsid, when used to package CMV-GFP independently, displayed a significantly increased tropism to the HEK293T cells (B″). In a second experiment, we used the BRAVE technology to improve the infectivity of primary cortical rat neurons in vitro. Both AAV2-WT and the AAV-MNMnull vector displays very poor infectivity of primary neurons (D-D′) and the AAV-MNM001 displays some improvement (D″). Through the BRAVE screen, we identified a number of peptides clustering over a C-terminal region of the HSV-2 pUL1 protein (C) which when utilized alone in a novel AAV capsid (AAV-MNM002) improved the infectivity of primary neurons in culture dramatically (D′″). (E) Comparison between titers for AAV2 and AAV-MNM001/004/008/009/017/025/026. Titers are shown as genome copies/cell (no of HEK293T at time of transfection). Selected capsids had to be produced at least twice with the same production method to be compared with AAV2. Black lines represent mean values, the two groups differed significantly using two tailed t-test, p≤0.05.

FIG. 3 Characterization of the AAV-MNM004 capsid for retrograde transport in vivo

(A) In the bioinformatics analysis of the HSV pUL22 protein, a C-terminal region of the protein displayed reproducible transport to all afferent regions; Cortex, Thalamus and Substantia Nigra while not showing the same bias at the injection site in the striatum. (B-D) HMM clustering of all peptides displaying these properties revealed two overlapping consensus motifs (C). capsid structures respectively. (D) The AAV-MNM004 was compared in vivo to the parent AAV2 through the unilateral striatal injection of a scAAV|CMV-GFP vector with each of the two capsid structures. 5 wks post AAV-injection, the animals were sacrificed and sections stained for GFP using immunohistochemistry developed into a brown precipitation staining using the DAB-peroxidase reaction. While the AAV2-Capsid promoted efficient transduction at the site of injection it resulted in very little retrograde transport of the vector. The AAV-MNM004 capsid on the other hand promoted a retrograde transport to all afferent regions as far back as the medial enthorhinal cortex.

FIG. 4 Utilization of the BRAVE approach to map and understand the function of proteins involved in Alzheimer's disease, both in vivo and in vitro

(A) Interestingly, the sAAP region shared significant sequence homology with a region of the VP1 protein from the Theiler's murine encephalomyelitis virus (TMEV) with appears to drive its axonal uptake and infectivity. (B-C, E) After deeper characterization, this region consisted of three adjacent conserved motifs with the third motif sharing significant homology with moth the VSV-G glycoprotein (well used to pseudotype lenti-viruses to improve neuronal tropism) and the HIV gp120 protein. Two novel capsid structures were generated from this region, AAV-MNM009 and AAV-MNM017. Both novel capsids promoted retrograde transport in vivo but AAV-MNM017 also displayed additional interesting properties. AAV-MNM017 infected both primary neurons and primary glial cells in vitro with very high efficacy. (D-D″) Detection in human primary glial cells. (D): AAV-MNM001 stained with mCherry; (D′): AAV-MNM017 stained with GFP; (D″): co-staining.

FIG. 5 Assessment of AAV capsid re-shuffling using BRAVE and generation of capsids infecting DA neurons

(A-B) In a last BRAVE screening experiment we aimed to develop a novel AAV capsid variant with efficient retrograde transport to dopamine neurons of the Substantia Nigra from injections into the Striatal output region. In this screening, we identified two regions of the CAV-2 capsid protein in close proximity. Interestingly, the first peptide shared significant homology with a third region of the same protein (A) while the second peptide (B) shared a peptide motif from the lectin soybean agglutinin (SA) which also efficiently transports from the synapse to the soma of neurons and can therefore be used as a retrograde tracer. (C-C′). Through double fluorescence immunohistochemistry we could then confirm that the majority of these cells were indeed TH-positive and thereby inferred to be DA producing (C″-C′″). Using the same in vitro hESC differentiation protocol we then assessed if the in vitro neuronal tropism of the de novo capsid variants would be maintained also on neurons with human origin. Indeed, all variants (MNM002, 008 and 010) which displayed high tropism on primary rodent neurons also showed much higher tropism than the wild-type AAV-variants.

FIG. 6 Functional dissection of the basolateral amygdala and its involvement in the development of anxiety

(A) In a last experiment, we utilized the BRAVE generated AAV-MNM004 capsid variant to answer an outstanding question regarding the functional contribution of the afferents from the basolateral amygdala to the dorsal striatum. This was conducted using a retrograde-induced chemogenetics (DREADD) approach. We injected the AAV-MNM004 vector expressing Cre-recombinase in the dorsal striatum and a Cre-inducible (DIO) chemogenetic (DREADD) vector into the basolateral amygdala (BLA) bilaterally into wild-type rats. (B) After selective induction of activity of the BLA neurons projecting to the dorsal striatum using the DREADD ligand CNO, we found a striking fear and anxiety phenotype here exemplified using the elevated plus maze (EPM) where the animals spent significantly less time on the open arms compared to control animals (where the Cre gene was replaced with GFP). This stands in stark contrast to the believed function of the BLA projections to the ventral striatum promoting positive stimuli. (C) This increases anxiety phenotype was accompanied by significant hypermobility in the open-field arena and a fear phenotype including excessive digging, severe sweating and episodes of freezing (D-E) after the CNO challenges, the animals were sacrificed and the BLA stained for either the HA-tag (identifying the hM3Dq DREADD expression) or mCherry (visualizing the rM3Ds DREADD) using immunohistochemistry developed into a brown precipitation staining using the DAB-peroxidase reaction.

FIG. 7 AAV production approaches

(A) 3-plasmid approach. The AAV genome is divided into two plasmids, a Transfer plasmid and a Packaging plasmid. The required genes from the Adenovirus (Ad) are then supplied in trans using a third, helper plasmid. The Transfer plasmid contains the genetic sequence to be packaged into the produced virions. This sequence is flanked by inverted terminal repeats (ITR) from AAV to be replicated and inserted into the capsid. This plasmid contains a gene of interest (GOI) driven by a Promoter and has a 3′untranslated region (3′UTR) and a poly-adenylation sequence (pA). The packaging plasmid contains the remaining parts of the wild-type AAV genome i.e., the Rep and the Cap genes usually driven by a strong promoter to increase titers. As these genes are no longer flanked by ITR sequences they are not packaged in the final AAV virions. The Helper plasmid contains the Ad E4, E2a and VA genes which, together with the Ad genes E1a and E1b, which may already expressed in the production cell line, allow for the AAV production. (B) 2-plasmid approach. The transfer plasmid is as in (A) but the Helper and packaging plasmids were merged into one larger plasmid. This retains the ability to produce replication deficient AAV-viruses using fewer plasmids. (C) Alternative 2-plasmid approach. The helper plasmid is as in (A) but the Transfer and Packaging plasmids are merged into one functional plasmid, providing both the Rep/Cap functionality and the ITR-flanked genome to be inserted into the AAV virion while the AAV vector is still replication deficient. This allows for the utilization of a much smaller amount of Transfer/packaging plasmid while maintaining titers as the Helper plasmid is rate limiting. It also ensures a perfect matching between the Cap gene and the GOI packaged inside.

FIG. 8 Cre-recombinase modulated readout

A two-factor selection regime may be used for in vivo selection of novel AAV capsid variants, which is exemplified here. The first factor is the site of delivery, e.g., systemic, intraventriular injection or intraparenchymal injection into a specific neuronal nucleus of choice. The second factor is a recombinase such as Cre recombinase or a DNA or RNA modifying protein such as Cas9, Cas13 or CPF1. This protein can be supplied either through the generation of transgenic animals or via viral vector. As a viral vector this can be delivered in a specific secondary brain nucleus to label only select afferents for capsid screening. The approach here shows a novel strategy that allows for on-target and off-target mapping based on barcodes sequenced from mRNA. The value of mRNA sequencing is that only successful infectivity results in mRNA formation and thus false-positives (non-infective particles retained in tissue) are excluded. (1) The delivered viral vector library contains a genome with the following key components. i) A molecular barcode (BC) which enables identification of the capsid structure based on an in vitro look-up table. ii) A unique sequencing primer binding site (SPBS) which enables enrichment and amplification of the library-derived mRNA for sequencing. iii) A synthetic polyadenylation site (spA) which terminates transcription only in the forward direction. iv) A Marker gene for on-target selection in the case of a low-abundance target. v) Two pairs of non-cross-compatible loxP recombination sites which provide an irreversible re-orientation in the presence of Cre-recombinase. vi) A unique 5′ untranslated region (5′UTR) which enables selective amplification of the barcodes from mRNA for sequencing in off-target cells. vii) A 3′UTR sequence for the same type of amplification in the on-target cells. (2) In the absence of Cre-recombinase, i.e., after off-target infectivity, the barcodes can be recovered and sequenced from mRNA using primers targeting the 5′UTR and the SPBS sites. This enables mapping of capsid variants with broad, non-selective infectivity. (3) When a virion infects a cell of interest, i.e., a Cre-expressing cell, recombination occurs between the two pairs of loxP sites. This results in a reversal of the orientation of the Marker gene, barcode and the SPBS sequence. (4) In the on-target cells the expressed mRNA allows for the translation of the Marker gene into protein but retains the SPBS and barcode as part of the mRNA 3′UTR thanks to the spA sequence not being active in the reverse orientation. From this mRNA, the barcodes can be selectively enriched and amplified using primers targeting the SPBS and the 3′UTR sequence.

### DETAILED DESCRIPTION OF THE INVENTION

The present disclosure provides a rationalised, systematic approach to design and manufacture a library of modified viral vectors encoding modified viral particles, where the modified viral particles comprise a modified capsid displaying a polypeptide fragment of selected proteins. Using tailored screening, fragments of said proteins which are useful for conferring a desired property to a viral particle can be identified. These can be used to design modified capsids, i.e. capsids displaying one of said identified fragments, with tailored properties. As can be seen in the examples, the methods can be used for example to design viral particles with increased tropism for a given cell type.

### Definitions

Expression: The term ‘expression’ of a nucleic acid sequence or polypeptide encoding a polypeptide refers to the transcription of that nucleic acid or polypeptide sequence as mRNA and/or transcription and translation of that nucleic acid sequence or polypeptide resulting in production of the protein encoded by the polynucleotide.

Gene therapy: The term ‘gene therapy’ used herein refers to the insertion of genes into an individual's cells and tissues to treat a disease.

Insertion: The term “insertion” is herein used to refer to polynucleotides or polypeptides which are inserted in a capsid gene or protein, respectively. A polynucleotide inserted in a capsid gene is inserted at a given position, “in addition” to the capsid gene; no polynucleotide fragment of the parent capsid gene is replaced by the polynucleotide, and the length of the capsid gene in which the polynucleotide is inserted is thus equal to the length of the parent capsid gene plus the length of the inserted polynucleotide. Likewise, the length of a capsid protein displaying a given polypeptide is equal to the length of the parent capsid protein plus the length of the displayed polypeptide.

Modified: The term is herein used in reference to a viral particle, a viral vector, a capsid gene or a capsid. A modified capsid is a capsid which displays a polypeptide as identified by the screening methods described herein. The capsid may thus have properties which have been altered by the insertion of said polypeptide. By extension, a modified capsid gene refers to a capsid gene in which a polynucleotide has been inserted, encoding for a polypeptide which when displayed on the capsid potentially alters its properties. Likewise, a viral vector is modified if compared to the viral vector from which it is derived it comprises an additional polynucleotide sequence encoding for a polypeptide which when displayed on the viral capsid may alter the capsid properties. A viral particle is modified if it comprises a modified capsid.

Operably linked: The term ‘operably linked’ as used herein when referring to two polynucleotides indicates that identification of one of the two polynucleotides enables identification of the other of the two polynucleotides. The two polynucleotides that are operably linked may be physically part of the same nucleic acid molecule, or they may be on different nucleic acid molecules, i.e. they may be operably linked in trans.

Promoter: The term ‘promoter’ used herein refers to a region of DNA that facilitates the transcription of a particular gene. Promoters are typically located near the genes they regulate, on the same strand and upstream.

Transgene: The term herein designates a polynucleotide, which it is desirable to introduce in a host cell or a target cell, and which is not naturally or natively expressed in said cell.

Viral genome: The term herein refers to the polynucleotide regions (DNA or RNA) which are flanked by terminal repeats (TR) and consequently packaged within a virion. For DNA viruses, the terminal repeats are inverted and termed inverted terminal repeats (ITR). Retroviruses and lentoviruses typically have long terminal repeats (LTR). Accordingly, a gene such as a capsid gene, which is outside the viral genome, may be on the same polynucleotide molecule as the viral genome, but is not flanked by TR sequences and is thus not packaged within the virions.

### Library of Viral Vectors or Particles

The present inventors have developed a method for manufacturing a library of viral vectors or viral particles, from which viral vectors or viral particles having a desired property can be selected. The method is based on the selection of a number of candidate polypeptides known to have or suspected of having a desired property, and identification of fragments thereof which, when displayed on a viral particle, confer the desired property to the thus modified viral particle.

Using the present methods, it is thus possible to design viral vectors encoding viral particles having a desired property, for example viral particles with improved tropism, or viral particles selectively targeting a specific type of cells. Such viral particles may be useful for a number of applications, e.g. gene therapy, particularly gene transfer to the central nervous system (CNS) and drug screening.

Accordingly, herein is provided a method of manufacturing a library of viral vectors comprising the steps of:
i) selecting one or more candidate polypeptides from a group of polypeptides having or suspected of having a desired property, and retrieving the sequences of said polypeptides;
ii) providing a plurality of candidate polynucleotides, each candidate polynucleotide encoding a polypeptide fragment of one of said candidate polypeptides, such that upon transcription and translation each candidate polypeptide is represented by one or more polypeptide fragments of each candidate polypeptide;
iii) providing a plurality of barcode polynucleotides;
iv) inserting each candidate polynucleotide together with a barcode polynucleotide into a viral vector, comprising a capsid gene and a viral genome, thereby obtaining a plurality of viral vectors each comprising a single candidate polynucleotide operably linked to a barcode polynucleotide, wherein the candidate polynucleotide is inserted within the capsid gene, the capsid gene is outside the viral genome and the barcode polynucleotide is inserted within the viral genome; wherein the viral vector comprises a marker polynucleotide encoding a detectable marker;
v) amplifying the plurality of viral vectors obtained in step iv) in an amplification system, wherein each viral vector is present in a plurality of copies in the amplification system; and
a) retrieving and transferring at least a first part of the plurality of viral vectors from the amplification system of step v) in a reference system, thereby mapping each barcode polynucleotide to one candidate polynucleotide; and
b) maintaining a second part of the plurality of viral vectors in the amplification system, and optionally transferring all or part of said second part in a production system to obtain a plurality of viral particles.

Also provided is a library of viral vectors, each viral vector comprising:
i) a backbone for expressing the viral vector in a host cell;
ii) a capsid gene and a candidate polynucleotide inserted therein, said candidate polynucleotide encoding a polypeptide fragment of a candidate polypeptide;
iii) a marker polynucleotide; and
iv) a barcode polynucleotide;
wherein
the candidate polypeptide is selected from a predefined group comprising one or more polypeptides having or suspected to have a desired property;
wherein upon transcription and translation each candidate polypeptide is represented by one or more polypeptide fragments in the library;
the candidate polynucleotide is inserted within the capsid gene of the viral vector so that it can be transcribed and translated to a polypeptide fragment displayed on the capsid, and is operably linked to a barcode polynucleotide inserted within the viral genome,
and the marker polynucleotide is comprised within the viral genome and the capsid gene is outside the viral genome.

### Candidate Polypeptides and Polynucleotides

In a first step, one or more candidate polypeptides are selected and their sequences are retrieved. The candidate polypeptides are polypeptides which are expected or suspected to confer a desired property to a viral particle when displayed on the capsid surface. The one or more candidate polypeptides may be one polypeptide, for instance if one desires to map the functional domains of the polypeptide with the methods described herein below, or it may be several polypeptides, as detailed below.

The candidate polypeptides may thus be known to be or suspected of being responsible for a given property. For example, in order to select polypeptides which potentially confer increased tropism toward a given type of cells when displayed on a capsid, a first polypeptide known to be transported to said type of cells may be selected. The remaining candidate polypeptides may be identified by running a blast query to identify other polypeptides, potentially from other entities, sharing motifs with the first polypeptide. The term “entity” should here be construed in the broadest sense and encompasses living organisms as well as viruses, prions and the like. Alternatively, all polypeptides from a given entity known to have or suspected of having said desired property at least in some conditions may be selected. The sequences of the selected candidate polypeptides are retrieved, by methods known to the skilled person. In the event that the sequences of the candidate polypeptides are not known, methods are available to the skilled person to determine said sequences.

Alternatively, the candidate polypeptides may originate from a peptide library, such as a synthetic library, for example a random peptide library. In some embodiments, the candidate polypeptides are not derived from the viral vector on which they are to be displayed. In some embodiments, the candidate polypeptides are derived from a mutant library; in a particular embodiment, the mutant library does not comprise mutants derived from a polypeptide which is native to the viral vector on which the polypeptides are to be displayed.

In a next step, a plurality of candidate polynucleotides is provided. The candidate polynucleotides encode fragments of the candidate polypeptides. The plurality of candidate polynucleotides may be ordered from a commercial provider, or designed and synthesised by the user. For instance, the sequence of the candidate polynucleotides may be drawn on paper or in silico, and order from a commercial provider, or synthesised by methods known in the art, for example on an array, as shown in example 1.

In some embodiments, the sequences of the candidate polynucleotides are codon-optimised for transcription in a given host cell, as is known in the art.

Each candidate polypeptide is represented by one or more polypeptide fragments each encoded by a candidate polynucleotide. In some embodiments, each candidate polypeptide is represented by at least two overlapping polypeptide fragments encoded by at least two polynucleotides, such as at least three overlapping polypeptide fragments encoded by at least three polynucleotides. It follows that the polynucleotides encoding the polypeptide fragments also overlap. As will be obvious to the person of skill in the art, the number of polypeptide fragments for different candidate polypeptides may be different. This is simply because it may in some cases be more convenient to design candidate polynucleotides that all have the same length, the number of candidate polynucleotides encoding overlapping polypeptide fragments of the same polypeptide thus being a function of the length of the corresponding candidate polypeptide. In other embodiments, the number of polypeptide fragments per candidate polypeptide may be the same for all candidate polypeptides, but their lengths may be different.

In some embodiments, it may be desirable to span all possible polypeptide fragments of a given length for a given candidate polypeptide. In such embodiments, the candidate polynucleotides are designed in such a way that all the candidate polynucleotides encoding polypeptide fragments of a same candidate polypeptide overlap over at least some of their length, such as over at least one codon. For maximal diversity to be reached, candidate polynucleotides encoding polypeptide fragments of a same candidate polypeptide preferably overlap but for one codon, so that all polypeptide fragments of a same candidate polypeptide overlap but for one amino acid residue. Such an approach is illustrated in the examples.

In other embodiments, the candidate polynucleotides encoding polypeptide fragments of a same candidate polypeptide overlap but for two codons, three codons, four codons, five codons, or more. Preferably, candidate polynucleotides encoding polypeptide fragments of a same candidate polypeptide overlap over at least one codon, such as at least two codons, such as at least three codons.

In some embodiments, the polypeptide fragment has a length of between 5 and 36 amino acid residues, such as between 5 and 30 amino acid residues. In one embodiment, the polypeptide fragment has a length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 residues.

In some embodiments, the polypeptide fragments all have the same length. In other embodiments, the polypeptide fragments have different lengths.

The polynucleotides encoding the polypeptide fragments of candidate polypeptides are to be inserted in the viral vector encoding the viral particle on which the polypeptide fragment is to be displayed; preferably the candidate polynucleotide is inserted within the capsid gene. Preferably, the capsid gene is outside the viral genome, i.e. it is not flanked by TR or ITR sequences. The resulting modified capsids thus each display a polypeptide fragment. Capsids have been studied thoroughly for a long time and the skilled person will have no difficulty in identifying suitable positions for inserting a polypeptide fragment to be displayed on the capsid.

As the skilled person is aware of, in embodiments where the viral particle is an AAV, the insertion site is preferably outside the lipase domain of VP1. It should also preferably be outside the assembly-activating protein (AAP). The insertion site may be at the N-terminus of VP2. It may also be at the vertices of the assembled capsid, e.g. centered around amino acid residue 587 of the Cap gene of AAV2 or around amino acid residue 588 of the AAV9 cap gene.

In preferred embodiments, the capsid displaying the candidate polypeptides has not otherwise been modified, i.e. its amino acid sequence is otherwise identical or essentially identical to the native or wild-type capsid. Accordingly, the length of the capsid protein displaying the polypeptide is in some embodiments always greater than the length of the native, unmodified capsid protein. In such embodiments, no polypeptide fragment of the native capsid is replaced by the candidate polypeptide, and all residues of the native capsid are also present in the modified capsid displaying the candidate polypeptide.

In embodiments where the viral vector is AAV2, the polynucleotides encoding the polypeptide fragments of candidate polypeptides may be for example designed so that the resulting polypeptide fragment is inserted between residues N587 and R588 of the VP1 capsid protein.

### Barcode Polynucleotides

Once the number of polypeptide fragments, and hence of candidate polynucleotides, has been determined, a plurality of barcode polynucleotides is provided. The barcode polynucleotides are unique, as will be detailed below. It will be recognised by the skilled person that the barcode polynucleotides preferably have a sequence which is not found in any of the candidate polynucleotides. The barcode polynucleotides should also preferably not be naturally present in the cell which is to be used as a production system, in order to avoid background noise in later steps. Additionally, the barcode polynucleotides should also not be naturally present in the host cells in which the library is to be expressed and screened in the methods of the present disclosure. The minimal length of the unique barcode polynucleotides will depend on the number of candidate polynucleotides, as will be evident to the skilled person. Each candidate polynucleotide is operably linked to a single barcode polynucleotide. Thus, the number of barcode polynucleotides is at least equal to the number of candidate polynucleotides or fragments. By “operably linked” is meant that each single candidate polynucleotide fragment is directly or indirectly connected to a single barcode polynucleotide, thereby also providing a link between each candidate polypeptide fragment and the corresponding unique barcode polynucleotide. Thus, the identification of a barcode allows identification of the corresponding polynucleotide fragment or polypeptide fragment. No barcode polynucleotide fragment can be linked to two different candidate polynucleotides (and hence indirectly two different candidate polypeptide fragments).

Methods to synthesise barcode polynucleotides are known in the art. These can also be ordered from a commercial provider.

Once unique pairs of candidate polynucleotides and barcode polynucleotides have been obtained, each pair is inserted in a viral vector to obtain a plurality of viral vectors each comprising a single candidate polynucleotide operably linked to a barcode polynucleotide. The viral vector comprises at least a capsid gene, which may be provided outside the viral genome, or in trans. By “viral genome” is meant the part of the viral DNA which is packaged in viral particles which is located within the inverted terminal repeats (ITRs) delimiting the end of the DNA molecule, or the part of the viral RNA which is packaged in viral particles, located within terminal repeats (TR) such as long terminal repeats (LTR) delimiting the end of the RNA molecule. The viral vector may also comprise a rep gene, which may also be provided in trans. The capsid gene (cap) and/or the rep gene may thus be provided in a packaging plasmid or as part of a helper plasmid (FIG. 7).

The pairs of candidate polynucleotides and barcode polynucleotides may be inserted in the viral vector simultaneously or sequentially. As explained above, since the method aims at screening modified capsids displaying polypeptide fragments in order to identify polypeptides conferring a desired property to a viral particle, the candidate polynucleotide is preferably inserted within the capsid gene, which is outside the viral genome. By contrast, the barcode polynucleotide is preferably inserted within the viral genome, i.e. between the terminal repeats, such as long terminal repeats or inverted terminal repeats. Without being bound by theory, this design avoids clonal enrichments and removes the bias introduced by PCR, which may be sequence-dependent. By sequencing the barcodes from RNA, the potential PCR bias is reduced and independent from the sequence of the modified capsid. Also, copy count per barcode does not influence the readout.

The barcode polynucleotide may be under the control of a promoter, as described below for the marker polynucleotides. Preferably, the barcode polynucleotide is introduced in the viral genome so that it can be transcribed, and optionally translated, when the viral particle has infected a cell.

It will be clear from the above that even though the barcode polynucleotide and the candidate polynucleotide are operably linked, they need not be contiguous on a same nucleic acid molecule.

### Marker Polynucleotide

The viral vector comprises a marker polynucleotide which encodes a detectable marker. The detectable marker enables monitoring of the expression pattern of the viral particles.

The marker polynucleotide may be any polynucleotide which upon transcription yields a stable mRNA molecule which is not naturally produced in the host cells in which the library of viral vectors is to be introduced in order to identify the candidate polypeptides responsible for a desired property, as detailed below. In some embodiments, the marker polynucleotide may encode a detectable marker, such as a fluorescent marker, which can be visualised or otherwise detected upon expression. The marker polynucleotide may also in some embodiments be the barcode itself. This can be relevant, for example when it is desirable to identify viral vectors which can infect cells which express a recombinase system, by the methods described herein below. The recombinase system may be a Cre recombinase combined with loxP sites, a CRISPR/Cas system such as CRISPR/Cas9, CRISPR/Cas13 or CRISPR/Cpf1.

This is illustrated, by way of example, on FIG. 8, where the recombinase is a Cre recombinase. A viral vector (here an AAV vector) comprising a barcode (BC) sequence between two pairs of loxP sites in opposite directions is shown. The region comprised within the loxP sites also comprises a binding site for a universal sequencing primer (SPBS). As is known in the art, if such a vector is transcribed in a cell expressing Cre recombinase, recombination between loxP sites will result in inversion of the sequence comprised therebetween. By contrast, in the absence of Cre expression, there will be no recombination and no inversion. The two events can be discriminated at the mRNA level by sequencing with a primer binding to SPBS and a primer binding to the 5′UTR or a primer binding to the 3′UTR. By sequencing the barcodes with primers binding to the 5′UTR and to SPBS, capsid variants having broad, non-selective infectivity can be mapped.

A polyadenylation site may be inserted downstream of the marker gene, as exemplified in FIG. 8, in such a way that transcription is only terminated if the polyadenylation site is in the forward transcription, i.e. transcription is only terminated if there is no recombination, in the absence of Cre recombinase. Transcripts issued from such cells will thus lack a barcode. By contrast, if Cre is expressed, transcription is not terminated, and the transcripts include a barcode. Sequencing of the transcripts thus allows discriminating between transcripts originating from cells expressing Cre recombinase and transcripts originating from cells lacking Cre recombinase expression.

The Cre recombinase may be provided in the cell via a vehicle such as a plasmid. It may also be comprised within the viral vector itself. Only the cells actually infected with the corresponding viral particle will thus express the Cre recombinase. The Cre recombinase may also be expressed by the host itself, for example when screening the library in a transgenic animal.

It is to be understood that the box marked “marker gene” on the figure is optional—as explained above, the barcode itself may serve the function of marker. In embodiments where a marker is present which is different from the barcode, it is to be noted that expression of the marker may require recombination to happen, as exemplified on the figure, so that the marker is downstream of the promoter and in the right direction.

In some embodiments, the marker polynucleotide encodes a marker polypeptide. The marker polypeptide may be selected from the group consisting of: a fluorescent protein, a bioluminescent protein, an antibiotic resistance gene, a cytotoxic gene, a surface receptor, β-galactosidase, the TVA receptor (the cellular receptor for subgroup A avian leukosis virus), pro-mitotic/oncogenes, trans-activators, transcription factors and Cas proteins. Numerous examples of suitable marker polypeptides or polynucleotides are known to the skilled person.

In some embodiments, the barcode polynucleotide is different from the marker polynucleotide and is located at the 3′ untranslated region (3′-UTR) of the marker polynucleotide. The barcode polynucleotide may, even if not used as main marker, still serve the function of additional marker polynucleotide.

In some embodiments, the marker polynucleotide is codon-optimised based on the codon preferences of the target cells in which the library of viral vectors is to be screened.

The marker polynucleotide may be under the control of a promoter. Accordingly, in some embodiments, the marker polynucleotide further comprises a promoter sequence. The promoter may be a constitutive promoter or an inducible promoter. When the barcode polynucleotide is not the marker polynucleotide, the barcode polynucleotide may be under the control of the same promoter as the marker polynucleotide.

The marker polynucleotide may be oriented in such a way relative to the promoter that transcription is only possible if the cell expresses a recombinase system, as explained above. The marker polynucleotide may comprise a polyadenylation site, which may be oriented in such a way that it terminates transcription only in one direction, as explained above for FIG. 8.

The choice of the promoter may be directed by the nature of the desired property which one wishes to screen the library for. Examples of promoters are: phosphoglycerate kinase (PGK), chicken beta actin (CBA), cytomegalovirus (CMV) early enhancer/chicken β actin (CAG), hybrid CBA (CBh), neuron-specific enolase (NSE), tyrosine hydroxylase (TH), tryptophan hydroxylase (TPH), platelet-derived growth factor (PDGF), aldehyde dehydrogenase 1 family member L1 (ALDH1L1), synapsin-1, cytomegalovirus (CMV), histone 1 (H1), U6 spliceosomal RNA (U6), calmodulin-dependent protein kinase II (CamKII), elongation factor 1-alpha (Efia), forkhead box J1 (Fox.J1), or glial fibrillary acidic protein (GFAP) promoters. The CMV, H1, U6, CamKII, Efia, Fox.J1 and GFAP promoters are known to be suitable for expression of polynucleotides in the central nervous system.

### Viral Vectors

Viral vectors which are suitable for modification by the methods disclosed herein comprise vectors derived from an adeno-associated viral (AAV) virus, a retrovirus, a lentivirus, an adeno-virus, a herpes simplex virus, a bocavirus and a rabies virus. Preferably, the viral vector is derived from a virus which is suited for delivering a transgene to a target cell. The transgene may be a gene used in gene therapy methods, or it may be a gene encoding a product of interest which it may be desirable to produce in the target cell.

### Amplification System

Once the plurality of viral vectors has been constructed, it is introduced in an amplification system. This allows multiple copies of each viral vector to be produced. The amplification system is any cell population suitable for the purpose, as is known to the person of skill in the art. Preferably, the amplification system is a prokaryotic cell population, e.g. a bacterial system, e.g. Escherichia coli.

The amplification system can be used to maintain the library, i.e. it can be used to preserve a part of the library containing at least one of each of the viral vectors of the library, in such conditions that the viral vectors can be retrieved from the amplification system. For instance, cells of the amplification system containing the library can be frozen and stored at −80° C. Aliquots can be taken from the stored amplification system to further amplify the library, and optionally to retrieve the viral vectors by methods known in the art.

### REFERENCE SYSTEM

In order to determine how each candidate polynucleotide (and hence each polypeptide fragment) is operably linked to a barcode polynucleotide, a first part of the plurality of viral vectors is retrieved from the amplification system above and transferred to a reference system. The reference system is a cell population from which the viral vectors can be further analysed. Preferably, the reference system is a bacterial cell population. The reference system is used to map the correspondence between candidate polynucleotide and barcode polynucleotide. This mapping step can be performed in several ways, e.g. retrieving viral vectors from the reference system and subsequently sequencing a region of each viral vector, where the sequenced region preferably comprises at least the barcode polynucleotide and the candidate polynucleotide.

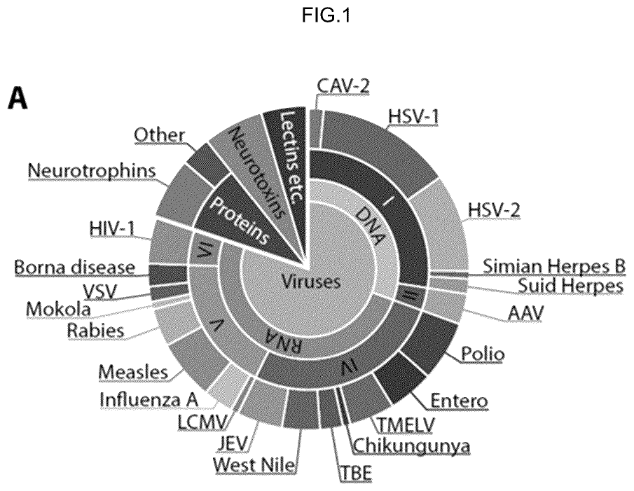

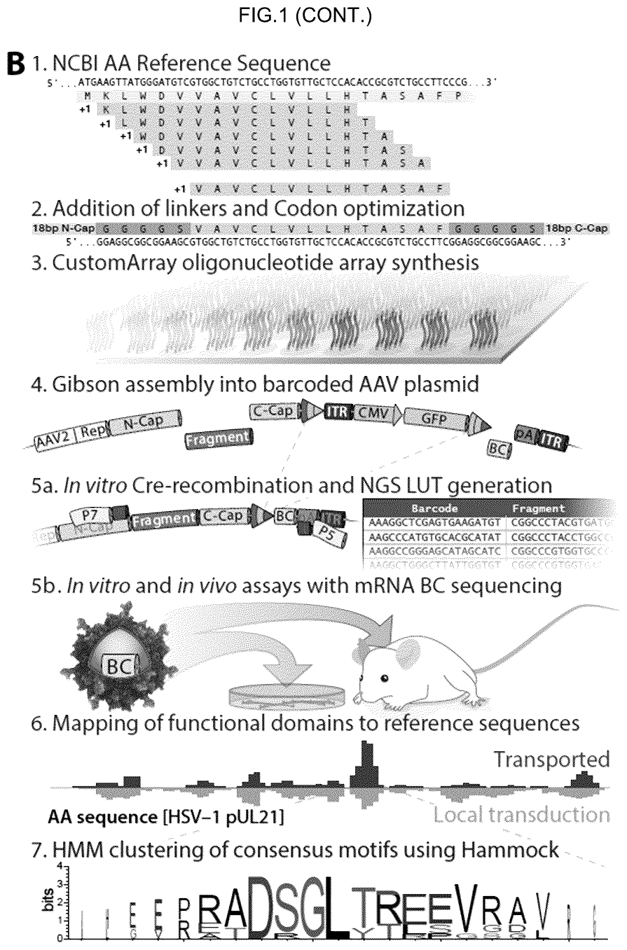

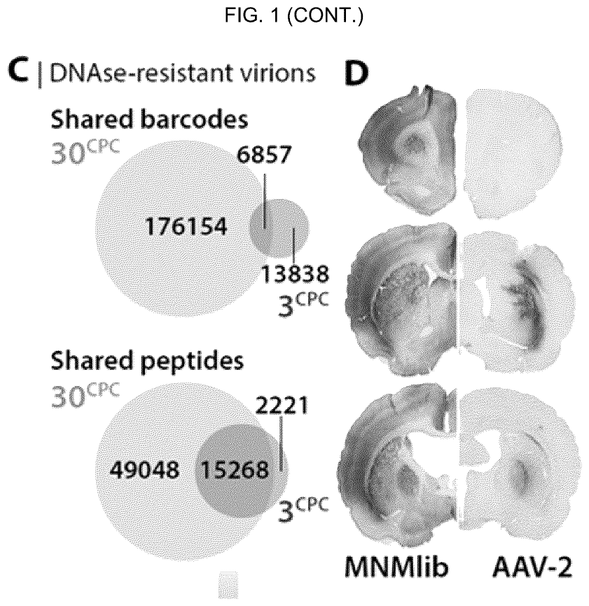

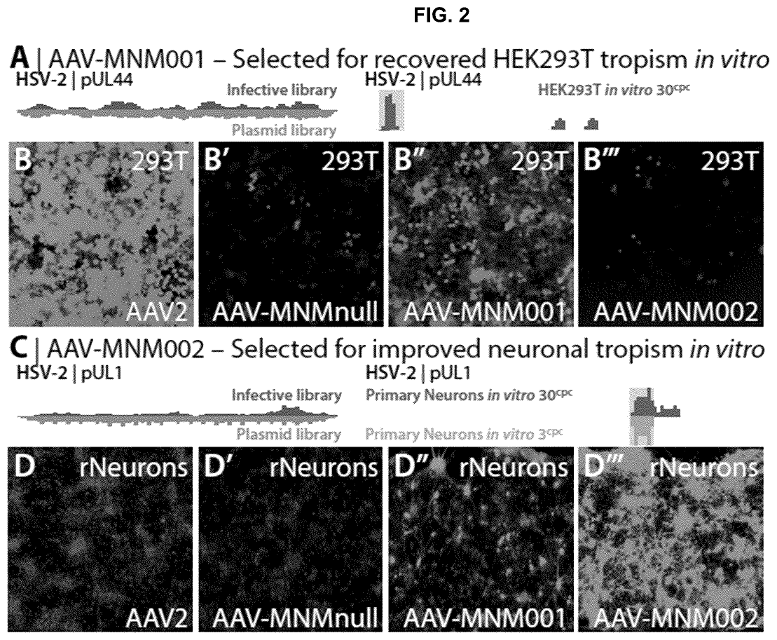

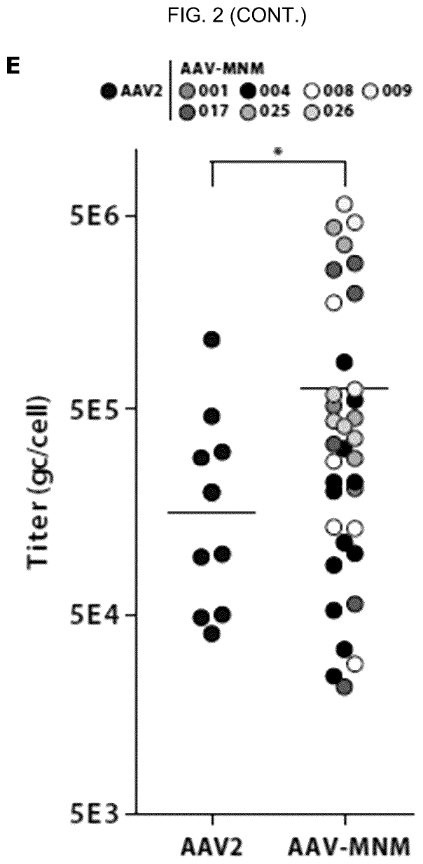

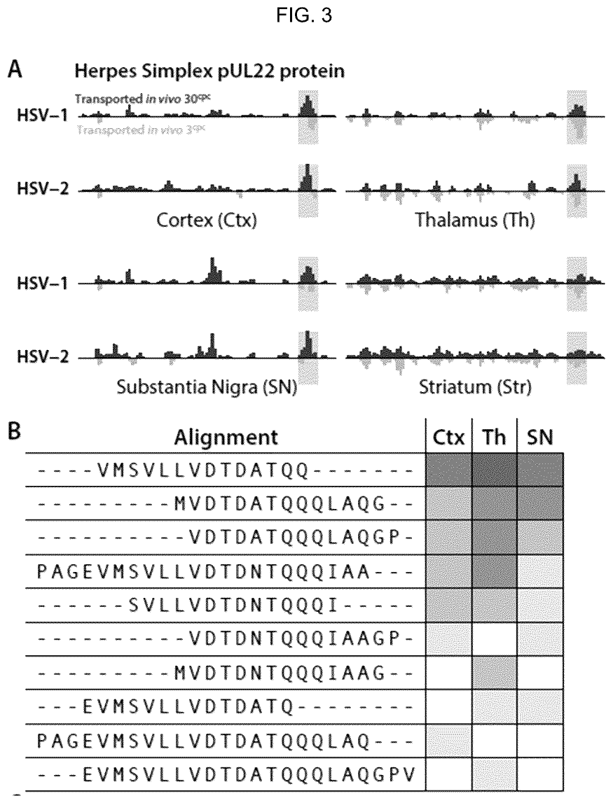

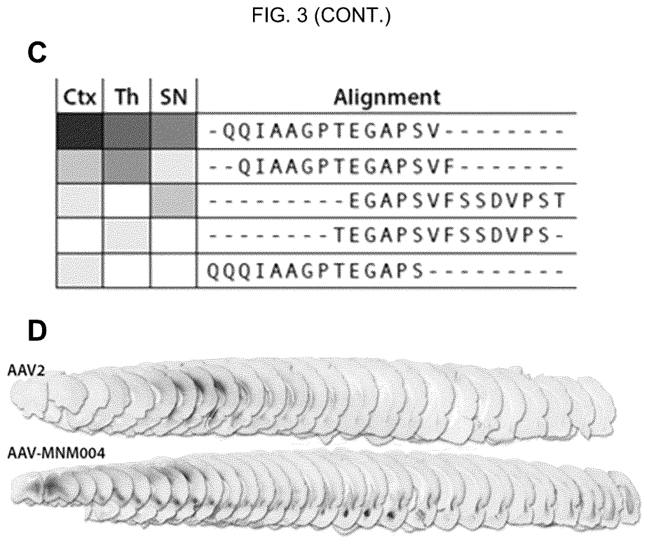

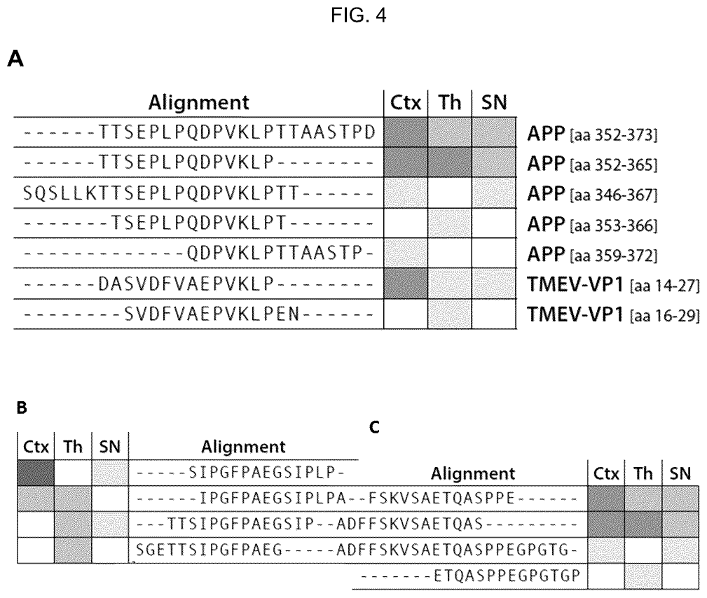

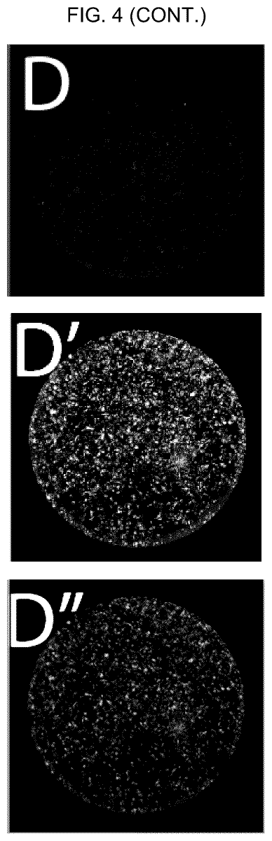

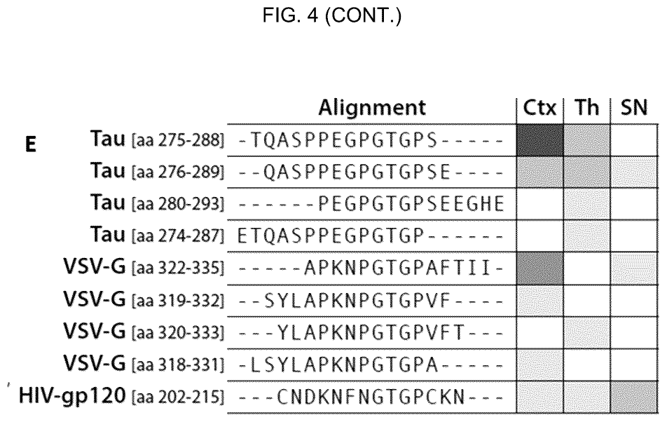

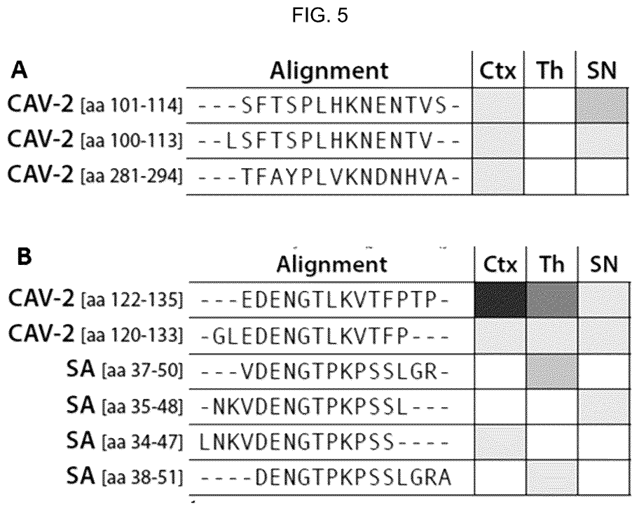

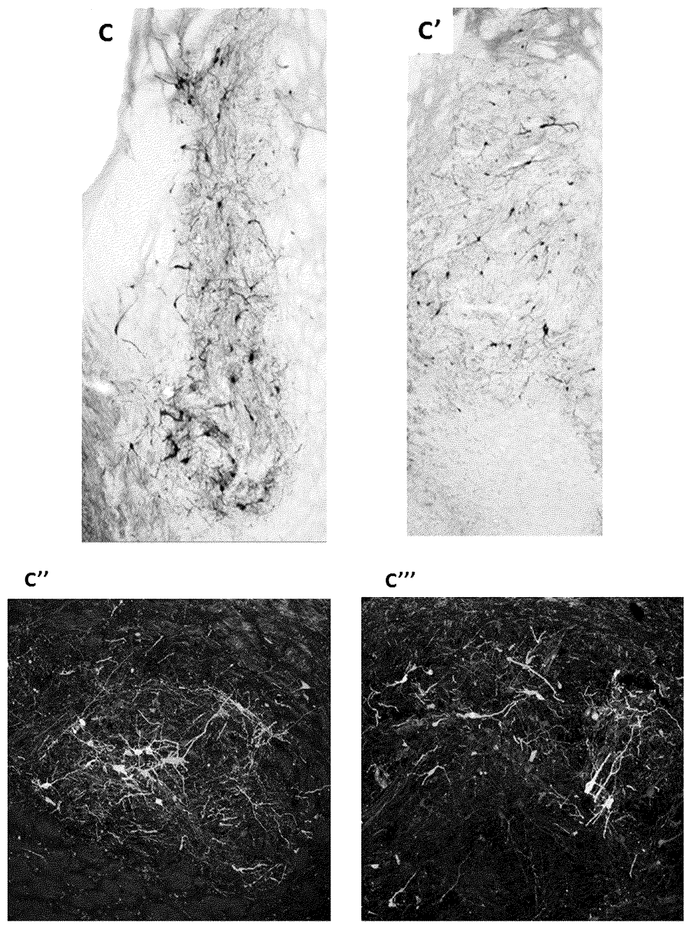

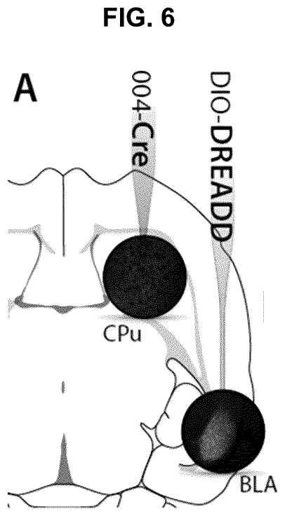

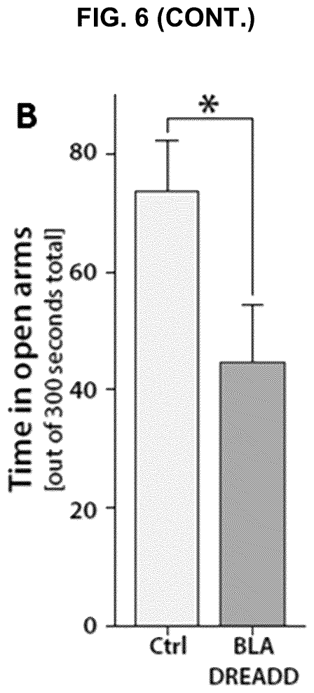

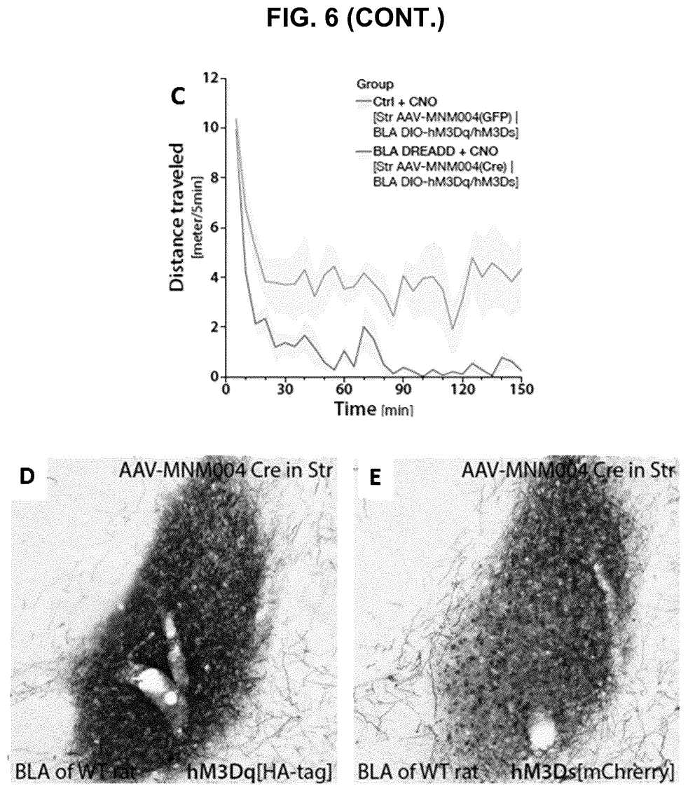

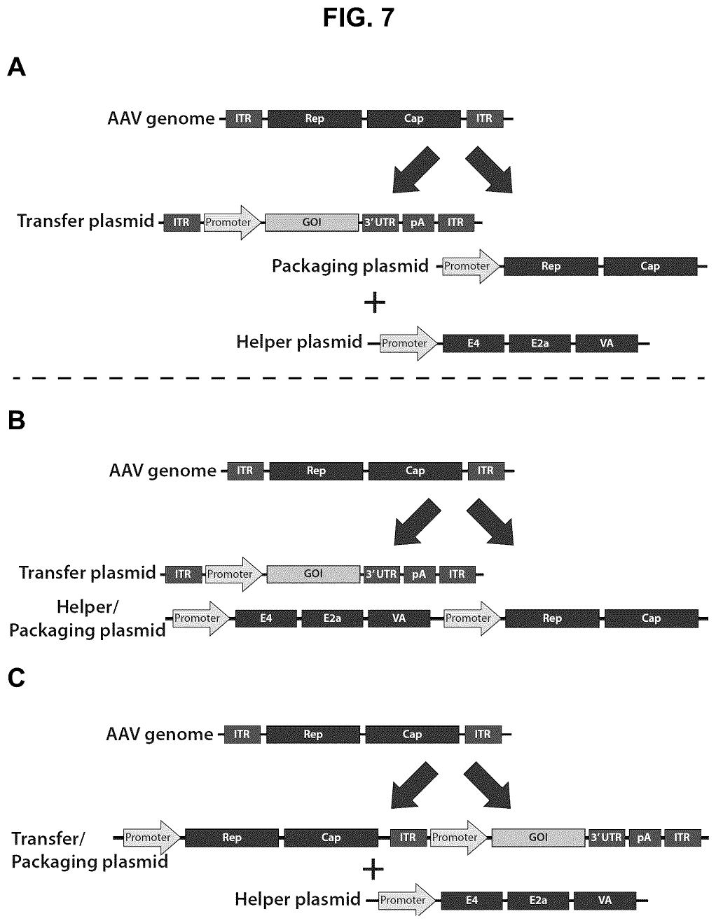

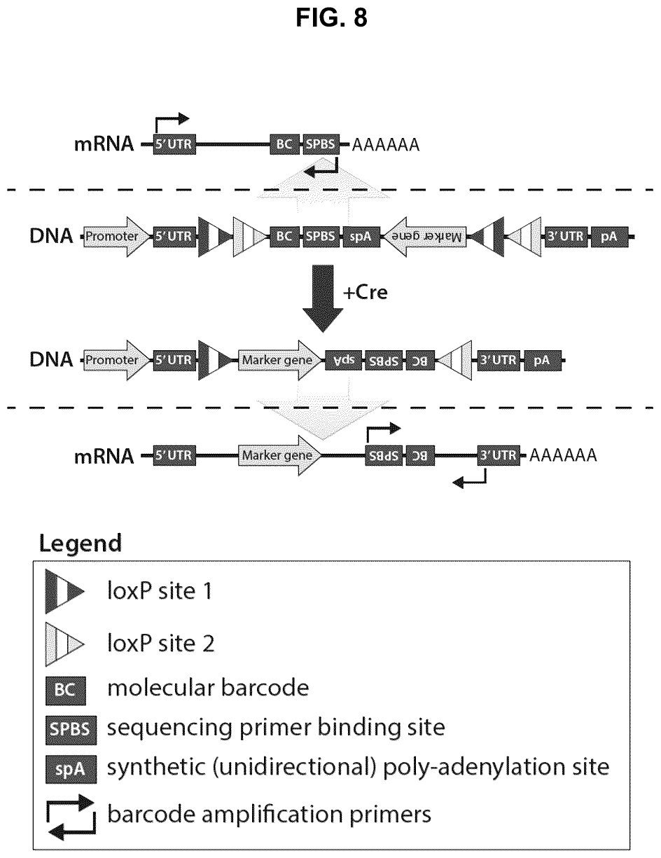

In some embodiments, a look-up table is generated, listing which barcode polynucleotide is linked to which candidate polynucleotide, and hence to which polypeptide fragment. An example of how to do this is illustrated in example 1 and FIG. 1B. After Cre-recombination followed by emPCR-based addition of Illumina sequencing adapters, the entire pool of candidate polynucleotides operably linked to random barcode polynucleotides was sequenced by paired-end sequencing, thereby obtaining a look-up table linking each polypeptide fragment to a unique barcode polynucleotide. Other methods to generate a look-up table will be evident to the skilled person.

Thus, the parallel use of an amplification system and a reference system allows the generation of viral vector preps simultaneously with the mapping of the correspondence between each barcode and each polypeptide fragment.

### Production System

In order to manufacture a library of viral particles from the library of viral vectors, a production system may be needed. The production system comprises a cell, and may further comprise plasmids or vectors comprising the elements necessary for the viral vectors to replicate and/or produce viral particles if these elements are not comprised within the viral vectors themselves, as is known in the art. Part of the plurality of viral vectors can thus be retrieved from the amplification system described above, so that said part comprises at least one of each of the viral vectors of the library, and can subsequently be transferred into a production system to obtain a plurality of viral particles.

The production system may comprise or consist of a mammalian cell, for example a human cell, an insect cell such as an SF9 cell, or a yeast cell such as a Saccharomyces cerevisiae cell. In some embodiments, the mammalian cell is a Hela cell, a primary neuron, an induced neuron, a fibroblast, an embryonic stem cell, an induced pluripotent stem cell, or an embryonic cell, such as an embryonic kidney cell, for example HEK293 cells.

The production system may further comprise vectors, e.g. plasmids, required for producing the viral particles in the cell. FIG. 7 shows several such plasmid systems exemplified for production of a DNA virus. Typical systems are based on three plasmids:
A transfer plasmid containing the genetic sequence packaged into the produced virions. The sequence is flanked by inverted terminal repeats to be replicated and inserted into the capsid. This plasmid may also contain a gene of interest driven by a promoter, a 3′ untranslated region (3′UTR) and a polyadenylation sequence.
A packaging plasmid, comprising the Rep and Cap genes, often under the control of a strong promoter.
A helper plasmid, which supplies the remaining genes required for viral production. In the case of an AAV, these may be E4, E2a and VA, and optionally E1a and E1b—some of these genes may however be expressed directly in the host cell.

Other systems based on two plasmids comprise a transfer plasmid as above, and one plasmid corresponding to both the helper and the packaging plasmid. Such systems allow production of replication deficient viruses in a simplified manner.

A third approach developed by the inventors is particularly suited for the screening methods disclosed herein. The packaging and the transfer plasmids are combined into one functional plasmid, which thus provides the Rep and Cap genes and the TR or ITR-flanked genome to be inserted in the virion, but still ensures that the vector is replication deficient. The helper plasmid is as described above, i.e. it supplies the remaining genes required for viral production. In particular embodiments, the production system thus comprises a cell, a plasmid providing the Rep and Cap genes and the TR or ITR-flanked genome, and a helper plasmid providing the remaining genes required for viral production.

### Diversity

Using the present methods, it is thus possible to design libraries having a high diversity, as illustrated in example 1. The diversity will typically increase proportionally to the number of polypeptide fragments of candidate polypeptides. It is also possible to further increase the library diversity by designing different polynucleotides for each polypeptide fragment. Likewise, polynucleotides encoding mutant polypeptide fragments, corresponding to the candidate polypeptides comprising one or more mutated residues compared to the native polypeptide, may also be included in the library.

In some embodiments, it may be desirable to obtain a library of viral particles with polypeptide fragments of a small subset of proteins, being as low as just one protein, in order to systematically map the functional domains of said protein. This approach was successfully used by the inventors to identify regions of APP and Tau, proteins known to be involved in Alzheimer's disease, which confer retrograde transport, as shown in example 3.

Designing and Manufacturing Viral Vectors and Viral Particles with a Desired Property

The present disclosure thus also provides methods for designing and manufacturing viral vectors with a desired property, said method comprising steps i) to v) as described herein above, and further comprising the steps of:
vi) retrieving a fraction of viral vectors from the amplification system of step v) b) above, or retrieving at least part of the viral particles from the production system of step v) b) above, and contacting a cell population with said retrieved viral vectors or viral particles;
vii) monitoring marker expression and selecting the cells wherein marker expression follows a desired pattern;
viii) identifying the barcode polynucleotides expressed in the cells selected in step vii), thereby identifying the candidate polynucleotides responsible for the desired property and the corresponding candidate polypeptides;
ix) designing a viral vector comprising a modified capsid gene, wherein the modified capsid gene comprises one of the candidate polynucleotides identified in step viii).

In some embodiments, the viral vector of step ix) is amplified in an amplification system, as described herein above.

In some aspects, the viral vector further comprises a transgene to be delivered to a host cell and is produced in a production system, thereby obtaining a viral particle having the desired property.

Also provided is a method of manufacturing a viral particle having a desired property, said method comprising steps i) to v) above, and further comprising the steps of:
vi) retrieving at least part of the plurality of viral vectors from the amplification system of step v) b) or retrieving at least part of the plurality of viral particles from the production system of step v) b);
vii) contacting a cell population with the retrieved viral vectors or viral particles obtained in step vi);
viii) monitoring marker expression and selecting the cells wherein marker expression follows a desired pattern;
ix) identifying the barcode polynucleotides expressed in the cells identified in step viii), thereby identifying the candidate polynucleotides responsible for the desired property and the corresponding candidate polypeptides;
x) designing a viral vector comprising a modified capsid gene, wherein the modified capsid gene comprises one of the candidate polynucleotides identified in step ix);
xi) producing the viral vector of step x) in an amplification system or in a production system, thereby obtaining the viral particle having the desired property.

Accordingly, are provided methods of identifying the candidate polypeptides responsible for a desired property, and using said candidate polypeptides to design and manufacture viral vectors and particles having a desired property.

### Desired Property

As will be evident from the examples below, the desired property may be any property which is desirable for a given application. The present methods thus allow identification, and subsequent design and production of corresponding viral vectors and viral particles, based on the identification of the candidate polypeptides which when introduced in a capsid confer one of more of the following properties:
affinity to a given cellular structure, such as a structure specific to a given type of cells, such as a synapse, or to a structure specific to a given cellular event, such as cellular division, cell differentiation, neuronal activation, inflammation or tissue damage;
improved transport properties, such as improved transport in the environment surrounding the host cell or improved transport across the blood-brain barrier;
increased ability to escape metabolism liver;
increased ability to evade the immune system;
increased ability to trigger the immune system.

The present methods can thus be used to identify polypeptide fragments which, when inserted in the capsid of a viral particle, can for example modify tropism of the particle, its infectivity, or its transport in the environment surrounding an injection site.

The inventors have, using the present methods and as illustrated in the examples, selected polypeptides known to have affinity to synapses to design a viral vector library. The library was then screened to identify the polypeptides which confer significantly improved infectivity compared to a mutated capsid having lost tropism for HEK293T cells in vitro. From the same library, modified capsids were identified having improved infectivity toward primary cortical neurons, or improved retrograde transport capacity.

The present disclosure thus also provides a method for improving a desired property to a viral particle comprising a capsid modified by insertion of said polypeptide therein, said method comprising steps i) to v) above, and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having an expression profile of the marker corresponding to an improvement of the desired property.

The reference viral vector or the reference viral particle may preferably be identical to the modified viral vector or modified viral particle, with the notable exception of the capsid. Preferably, the capsid of the reference viral vector or particle is not modified.

### Cell Population and Target Cells

In a subsequent step, the viral particle library is tested in a cell population. The nature of the desired property will be important for determining the nature of the cell population which is to be contacted with the plurality of viral particles of the library, in order to identify the particles which have the desired property. For instance, if the desired property is increased tropism toward specific types of cells, e.g. of the central nervous system, the cell population must comprise said specific types of cells.

The library of particles may be contacted with a cell population in vitro or in vivo. The library of viral particles may for example be injected in an animal or a human, for example at a specific injection site, or it may simply be contacted with a cell culture.

It is then possible to monitor marker expression and select the cells where marker expression follows the expected or desired pattern. This can be done by methods known in the art. For example, if a fluorescent marker is used, the cells may be monitored for fluorescent emission, following which RNA is extracted from the cells in order to identify the expressed barcodes. RNA may also just be extracted from the target cells, and the barcode polynucleotides may subsequently identified by sequencing.

Since the barcode polynucleotides are each operably linked to a single polypeptide fragment, identification of the barcode polynucleotide enables identification of the polypeptide fragments responsible for the desired expression pattern. Said polypeptide fragments may then be used to design viral vectors encoding modified viral particles with a modified capsid, by inserting the polynucleotide encoding the relevant polypeptide fragment in the capsid so that it is displayed on the surface of the particle, thereby altering the properties of the native capsid, as described herein above. The modified viral vectors or particles do not require the presence of the barcode polynucleotide. Alternatively, the viral vectors or viral particles displaying the desired properties may be retrieved directly from the cell population in which they are tested.

The modified viral particles may then be amplified in an amplification system and/or produced in a production system, as described herein above.

### Delivering a Transgene to a Target Cell

The methods disclosed herein may thus be used to design modified viral particles comprising a capsid modified by insertion of a polypeptide conferring a desired property, where the modified viral particles are particularly well suited for delivery of a transgene to a target cell.

By the term “transgene” is to be understood as referring to a polynucleotide comprising a gene isolated from one organism and to be introduced into a different organism. A transgene is thus not native to the target cell.

Accordingly, the present modified viral particles may encapsulate a transgene to be delivered to a target cell. Upon injection of the modified viral vector or viral particle into an injection site, or upon contact with a cell population comprising the target cell, the transgene is delivered to the target cell. It is to be noted that the target cell does not need to be in the vicinity of the injection site, provided that the modified viral vector or modified viral particle is capable of being transported to the target cell from the injection site. The term “injection site” should be understood in its broad sense, i.e. it may refer to a site of an organism to which the vectors or particles are injected, or it may simply refer to a cell population comprising the target cells which it is desirable to infect with the vectors or particles upon contact therewith.

Thus are also disclosed herein modified viral particles for delivering a transgene to a target cell, comprising a modified capsid gene and a transgene to be delivered to the target cell. Viral vectors encoding such viral particles are also disclosed.

Accordingly, the use of the modified viral particles disclosed herein, which may comprise a modified capsid as detailed further below, for a method of treatment comprising or consisting of a step of gene therapy is also provided.

Preferably, the modified viral particles present improved properties compared to an unmodified viral particle, i.e. a viral particle which comprises a native, unmodified capsid gene. The improved properties may be any of the properties listed herein above.

The modified viral particle may be derived from an adeno-associated virus (AAV), a retrovirus, a lentivirus, an adeno-virus, a herpes simplex virus, a bocavirus or a rabies virus.

The modified viral particle may comprise a modified capsid as disclosed herein, in particular a modified capsid comprising or consisting of a polypeptide which comprises or consists of a variant of SEQ ID NO: 1 to SEQ ID NO: 50, wherein at the most one amino acid residue has been deleted, modified or replaced. In other embodiments, the modified capsid comprises a polypeptide which is a variant of SEQ ID NO: 1 to SEQ ID NO: 50, wherein at the most two amino acid residues have been deleted, modified or replaced. In other embodiments, the modified capsid comprises a polypeptide which is a variant of SEQ ID NO: 1 to SEQ ID NO: 50, wherein at the most three amino acid residues have been deleted, modified or replaced. The variant may be a variant of of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.

### Transgene

The nature of the transgene will typically be directed by the result that is desired.

The transgene may be a gene useful for gene therapy, for example a “replacement” or “correction” gene replacing a gene which is deficient in an individual. The transgene may also encode a protein or a transcript which upon expression may compensate for a deficient mechanism in the target cell. The transgene may also be used to knock down or reduce expression of a gene causing a disease. This can be by way of inhibition if the transgene codes for a silencing RNA. Below are listed some examples of genes that may be targeted by transgenes using the present vectors to treat or alleviate symptoms of diseases of the nervous system, by way of illustration.

The transgene may be a gene involved in the synthesis of dopamine, which may be useful to alleviate symptoms of Parkinson's disease, for examples genes encoding tyrosine hydroxylase, aromatic amino-acid decarboxylase (AADC), GTP-cyclohydrolase 1 (GCH1) or vesicular mono-amine transporter 2 (VMAT2). The transgene may also be a neuroprotective gene, which it may be desirable to express for example in patients suffering from Parkinson's disease, such as Nurri, GDNF, neurturin (NRTN), CNDF or MANF. The transgene may upon expression result in knock-down or correction of genes causing Parkinson's disease, e.g. alpha-synuclein (SNCA), LRRK2, Pink1, PRKN, GBA, DJ1, UCHL1, MAPT, ATP13A2 or VPS35.

Examples of genes that may be knocked down or corrected in Alzheimer's patients are APP, MAPT, SPEN1 and PSEN2. In Huntington's disease patients, the HTT gene (coding for huntingtin) may be knocked down or corrected by delivery of the transgene. In patients suffering from spinocerebellar ataxia, ataxin 1, 2, 3, 7 or 10, PLEKHG4, SPTBN2, CACNA1A, IOSCA, TTBK2, PPP2R2B, KCNC3, PRKCG, ITPR1, TBP, KCND3 or FGF14 may be knocked down or corrected upon delivery and expression of the transgene. In multiple system atrophy, SNCA or COQ2 may be relevant targets of for knock-down or correction. In amyotrophic lateral sclerosis, the transgene may lead to overexpression or correction of C9orf72, SOD1, TARDBP or FUS. SMN1, SMN2, UBA1, DYNC1H1 or VAPB are targets for knock-down or correction in spinal muscular atrophy patients. DMD is a target for overexpression or correction in Duchenne muscular dystrophy. The transgene may allow for correction of ABCA13, C4A, DGCR2, DGCR8, DRD2, MIR137, NOS1AP, NRXN1, OLIG2, RTN4R, SYN2, TOP3B, YWHAE or ZDHHC8 in schizophrenic patients. Galanin, NPY, somatostatin or KCNA1 may be targets for overexpression in epileptic patients. The transgene may enable overexpression of p11, PDE11a, channel rhodopsins or chemogenetic receptors in individuals suffering from depression.

The transgene may in other embodiments be an immunogenic agent, e.g. the modified viral particle may be used to target cells of the immune system to immunise an individual to a given epitope.

Disclosed herein are modified viral particles comprising a modified capsid, wherein the modified capsid comprises or consists of a polypeptide comprising or consisting of a sequence selected from the group consisting of SEQ ID NO: 1 to SEQ ID NO: 50. Also disclosed are modified viral vectors encoding said modified viral particles. In one embodiment, the modified capsid comprises a polypeptide comprising or consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or SEQ ID NO: 50.

Such modified capsids may comprise any of the above polypeptide fragments, or variants thereof, i.e. modified polypeptide fragments, wherein at the most one, such as at the most two, such as at the most three amino acid residues have been deleted, modified or replaced.

Accordingly, in some embodiments, the modified capsid comprises a polypeptide which comprises or consists of a variant of SEQ ID NO: 1 to SEQ ID NO: 50, wherein at the most one amino acid residue has been deleted, modified or replaced. In other embodiments, the modified capsid comprises a polypeptide which is a variant of SEQ ID NO: 1 to SEQ ID NO: 50, wherein at the most two amino acid residues have been deleted, modified or replaced. In other embodiments, the modified capsid comprises a polypeptide which is a variant of SEQ ID NO: 1 to SEQ ID NO: 50, wherein at the most three amino acid residues have been deleted, modified or replaced. The variant may be a variant of of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.

In some embodiments, the polypeptide is encoded by a polynucleotide comprising or consisting of a sequence selected from the group consisting of SEQ ID NO: 51 to SEQ ID NO: 100. In some embodiments, the polypeptide is encoded by a polynucleotide comprising or consisting of a sequence selected from SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99 or SEQ ID NO: 100.

As is known in the art, the transgene may be inserted in the viral genome, i.e. between the terminal repeat sequences, such as long terminal repeats or inverted terminal repeat sequences, delimiting the viral genome.

### Target Cells

The cells to be targeted for transgene delivery are preferably cells in which expression of the transgene is desired. The target cells may for example be neurons, such as cortical neurons and/or neurons of the hippocampus and/or the entorhinal cortex and/or the cerebellum and/or of the spinal cord and/or at the epileptic focus and/or of the nucleus accumbens and/or of the Habenula; glial cells, in particular in the caudate-putamen and/or the substantia nigra and/or the cerebral cortex and/or of the infarct area; DA neurons, in particular of the substantia nigra and the ventral tegmental area; or muscle myocytes.

Libraries of modified viral vectors can be screened as described above, where the improved property that is selected for is increased infectivity and/or tropism for a target cell, in particular the target cells listed above.

### Methods of Treatment

Also provided herein is a modified viral vector as disclosed anywhere herein, or a modified viral particle as disclosed herein, for use in a method of treatment or prophylaxis of a disorder.

The modified viral particle displays a polypeptide which may for example result in increased infectivity of cells to be targeted for delivery of a transgene which may be useful for treating or preventing said disorder.

Accordingly, herein is disclosed a method of treatment or prophylaxis of a disease or disorder, comprising the steps of administering a modified viral vector or a modified viral particle as described herein to a subject in need thereof, said modified viral vector or particle comprising a transgene. Preferably the modified viral particle has increased tropism and/or infectivity for the target cell to which the transgene is to be delivered than a corresponding, unmodified viral particle with an unmodified capsid.

The modified viral particle may comprise a modified capsid as disclosed herein, in particular a modified capsid comprising or consisting of a polypeptide which comprises or consists of a variant of SEQ ID NO: 1 to SEQ ID NO: 50, wherein at the most one amino acid residue has been deleted, modified or replaced. In other embodiments, the modified capsid comprises a polypeptide which is a variant of SEQ ID NO: 1 to SEQ ID NO: 50, wherein at the most two amino acid residues have been deleted, modified or replaced. In other embodiments, the modified capsid comprises a polypeptide which is a variant of SEQ ID NO: 1 to SEQ ID NO: 50, wherein at the most three amino acid residues have been deleted, modified or replaced. The variant may be a variant of of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.

The subject in need may be a subject suffering from, suspected of suffering from, or at risk of suffering from a disease or disorder.

In some embodiments, the disease is Parkinson's disease. The transgene may encode tyrosine hydroxylase, aromatic amino-acid decarboxylase (AADC), GTP-cyclohydrolase 1 (GCH1) or vesicular mono-amine transporter 2 (VMAT2); preferred target cells for such embodiments are neurons and or glial cells in the caudate-putamen and/or the substantia nigra. The transgene may lead to overexpression of neuroprotective genes, such as Nurri, GDNF, neurturin (NRTN), CNDF or MANF; preferred target cells for such embodiments are neurons and or glial cells in the caudate-putamen and/or the substantia nigra. The transgene may lead to knock-down or correction of alpha-synuclein (SNCA), LRRK2, Pink1, PRKN, GBA, DJ1, UCHL1, MAPT, ATP13A2, VPS35; preferred target cells for such embodiments are DA neurons of the substantia nigra and the ventral tegmental area.

In other embodiments, the disease is Alzheimer's disease, and the transgene leads to knock-down or correction of APP, MAPT, SPEN1 or PSEN2. Preferred target cells for such embodiments are neurons of the hippocampus and the entorhinal cortex.

In other embodiments, the disease is Huntington's disease and the transgene knocks down or corrects HTT. Preferred target cells for such embodiments are neurons and or glial cells in the caudate-putamen and/or the cerebral cortex.

In other embodiments, the disease is spinocerebellar ataxia and the transgene knocks down or corrects ataxin 1, 2, 3, 7 or 10, PLEKHG4, SPTBN2, CACNA1A, IOSCA, TTBK2, PPP2R2B, KCNC3, PRKCG, ITPR1, TBP, KCND3, or FGF14. Preferred target cells for such embodiments are neurons of the cerebellum.

In other embodiments, the disease is multiple system atrophy and the transgene knocks down or corrects SNCA or COQ2. Preferred target cells for such embodiments are neurons and or glial cells in the caudate-putamen and/or the substantia nigra and/or the cerebral cortex

In other embodiments the disorder is amyotrophic lateral sclerosis and the transgene leads to overexpression or correction of C9orf72, SOD1, TARDBP or FUS. Preferred target cells for such embodiments are neurons of the spinal cord.

In other embodiments, the disorder is spinal muscular atrophy and the transgene leads to knock-down or correction of SMN1, SMN2, UBA1, DYNC1H1 or VAPB. Preferred target cells for such embodiments are neurons of the spinal cord.

In other embodiments, the disorder is Duchenne muscular dystrophy and the transgene leads to overexpression or correction of DMD. Preferred target cells for such embodiments are muscle myocytes.

In other embodiments, the disorder is schizophrenia and the transgene leads to correction of ABCA13, C4A, DGCR2, DGCR8, DRD2, MIR137, NOS1AP, NRXN1, OLIG2, RTN4R, SYN2, TOP3B, YWHAE or ZDHHC8. Preferred target cells for such embodiments are cortical neurons.

In other embodiments, the disorder is epilepsy and the transgene leads to overexpression of galanin, NPY, somatostatin or KCNA1. Preferred target cells for such embodiments are neurons at the epileptic focus.

In other embodiments, the disorder is depression and the transgene leads to overexpression of p11, PDE11a; preferred target cells for such embodiments are neurons of the nucleus accumbens; or the transgene leads to overexpression of channel rhodopsins or chemogenetic receptors; preferred target cells for such embodiments are neurons of the Habenula.

### Drug Screening

The modified viral vectors and particles obtainable by the methods disclosed herein may also be useful for a number of additional applications, including drug screening.

In one aspect is provided a method for identifying a drug having a desired effect, said method comprising the steps of:
a) Providing a candidate drug;
b) Administering the candidate drug to a cell;
c) Providing a modified viral particle comprising a modified capsid allowing delivery of the viral particle to the cell of b) and a marker polynucleotide;
d) Monitoring and comparing expression and/or localisation of the marker polypeptide in the presence and absence of the candidate drug;
thereby determining whether the candidate drug has an effect on the expression of the marker polynucleotide.

The modified viral particle may comprise a modified capsid as disclosed herein, in particular a modified capsid comprising or consisting of a polypeptide which comprises or consists of a variant of SEQ ID NO: 1 to SEQ ID NO: 50, wherein at the most one amino acid residue has been deleted, modified or replaced. In other embodiments, the modified capsid comprises a polypeptide which is a variant of SEQ ID NO: 1 to SEQ ID NO: 50, wherein at the most two amino acid residues have been deleted, modified or replaced. In other embodiments, the modified capsid comprises a polypeptide which is a variant of SEQ ID NO: 1 to SEQ ID NO: 50, wherein at the most three amino acid residues have been deleted, modified or replaced. The variant may be a variant of of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.

### Functional Mapping of Protein Domains

The modified viral vectors and particles obtainable by the methods described herein can be used to perform functional mapping of protein domains. This is illustrated in example 5.

By the present methods, polypeptide fragments of polypeptides known to be or suspected of being involved in a given mechanism or disorder may be used to manufacture a library of modified capsids, where the modified capsids display different regions of the polypeptides.

Accordingly is provided herein a method of identifying one or more regions of a polypeptide conferring a desired property to a viral particle comprising a capsid modified by insertion of said polypeptide therein, said method comprising steps i) to v) above and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having an expression profile of the marker corresponding to the desired property, thereby identifying the region of the polypeptide responsible for said property.

For such applications, as will be evident to the skilled person, the higher the number of polypeptide fragments displayed by the capsid library and/or the greater the overlap between the individual polypeptide fragments is, the more precise the functional mapping will be. Accordingly, in some embodiments, each polypeptide is represented by a number of polypeptide fragments in such a way that the polypeptide fragments overlap by all but one amino acid residue.

### EXAMPLES

### Example 1—Design and Generation of Highly Diverse AAV Library, and Screening for Retrograde Transport Function

### Materials and Methods

### AAV Library Backbone Plasmid Cloning

The backbone plasmid used for cloning the barcoded modified AAV capsids was developed from a self-complementary AAV (scAAV) vector expressing GFP (pscAAV-GFP24) and pDG25 (with deletions of Adenovirus genes VA, E2A and E4). The final plasmid contained an eGFP expression cassette driven by a CMV promoter and the wild type AAV2 genome with the mouse mammary tumor virus (MMTV) promoter. First, the XbaI, BsiWI and MluI sites were inserted between XhoI and HindIII sites in pscAAV-GFP [Addgene #32396]. Second, an Nhel site was introduced between sequences of N587 and R588 of VP1 capsid protein by overlap extension PCR26 using modified pDG as template. Besides the Nhel site, the final PCR product also contained a BsiWI and a MluI site to facilitate subsequent cloning and LoxP-JTZ17 insertion (for Cre recombination and Next Generation sequencing of the final library). Finally, the modified pscAAV-GFP was digested using XbaI and MluI, the overlap extension PCR product was digested with MluI and BsiWI, and pDG was digested by BsiWI and XbaI. The three DNA fragments were then ligated to acquire the final backbone plasmid for the AAV library.

### Selecting Proteins for Peptide Display

Candidates of peptides to be inserted were derived from known neuron-related proteins. 131 proteins were selected, belonging to five categories; neurotropic viruses, lectins, neurotrophins, neurotoxins, and neuronal proteins. The candidate protein selection was based on known interaction between the proteins and neurons in binding and different stages of AAV infection and replication process (e.g. internalization, endosomal trafficking, nuclear import, etc.). Peptides were designed to be incorporated between N587 and R588 of VP1 capsid protein 11, a site that previously was reported to tolerate insertion of large peptides ^{14, 27} and blocks heparan sulfate proteoglycan binding ^{12, 13}. Four different peptide conformations were designed as: A-14aa-A, A-22aa-A, A5-14aa-A5, G4S-14aa-G4S. They contained two lengths of peptides of 14 or 22 amino acid (aa) residues and were flanked by either a spacer of one amino acid of alanine (A) or a short linker (A5 or G4S) ^{28, 29}. All possible unique peptides of 14 or 22 aa from the candidate proteins were identified and generated by a sliding window approach using R program. The peptide library was reverse translated to oligonucleotides using codon optimization for high-level expression in HEK293 cells.

### Array Oligonucleotide Synthesis and Amplification

The final oligonucleotide pool containing 92,918 unique oligonucleotides was synthesized using 90 k DNA array (Custom Array), which encoded all possible unique peptides from A-14aa-A, and selected peptides from the other three peptide conformations.

The oligonucleotide pool was amplified and prepared for Gibson assembly in an emulsion PCR with long extension time to reduce PCR artifacts ^{17,30}.

PCR mix was prepared using Phusion™ Hot Start II High-Fidelity DNA polymerase (Thermofisher) according to manufacturer's recommendation with the exception of adding 0.5 μg/μl BSA (NEB). Briefly, 9 volumes of an oil surfactant mixture (92.95% of Mineral oil, 7% of ABIL WE and 0.05% of Triton X-100) was added to the PCR mixture and an emulsion was created by homogenizing for 5 min at a speed of 4 m/s using MP FastPrep-24 Tissue and Cell Homogenizer (MP Biomedicals). The PCR program used for the emulsion PCR was; 1 cycle of 30s at 98° C., 30 cycles of 5s at 98° C., 30s at 65° C. and 2 min (8-fold of regular extension time) at 72° C. and finally 1 cycle of 5 min at 72° C. After the PCR, the emulsion was broken by adding 2 volumes of isobutanol to each tube, the aqueous phase containing the PCR product was separated by a short centrifugation (16,000 g for 2 min) and finally purified using E.Z.N.A.™ Cycle Pure Kit (Omega).

### AAV Library Cloning and Barcoding

Gibson assembly was used to insert the oligonucleotide pool (into the capsid gene located outside of the ITR's) and barcodes (downstream of GFP located inside of the ITR's) to generate a barcoded AAV plasmid library (FIG. 1). A one cycle PCR was performed to generate barcoded fragments with overhangs for Gibson assembly. Barcode length was 20 nucleotides and defined as ambiguity nucleotides by using the sequence V-H-D-B (IUPAC ambiguity code) repeated five times and flanked by static sequences for binding. Oligos also contained a LoxP-JTZ17 site, for facilitating subsequent Cre recombination. A 40 μl Gibson Assembly reaction (NEB) was performed to insert oligonucleotide pool and barcoded fragments into 200 ng of digestedvector, using a molar ratio of 1.3:1.3:1. The reaction was incubated for 1 h at 50° C. and purified using DNA Clean & Concentrator-5 (Zymo Research). 1 μl (37.4 ng) purified Gibson assembly product was transformed into 20 μl MegaX DH10B™ T1R Electrocomp™ (Thermo Fischer Scientific) cells according to the manufacturer's protocol. 5 individual transformations were performed and pooled into one tube. A small fraction of the transformed bacteria was plated on agar plates to validate the transformation efficacy. 10 clones were picked from the plates and oligonucleotide and barcode insertion was validated by restriction enzyme digestion using Bsp120I, BsrGI, and SpeI (Fastdigest, Thermo Fischer Scientific). The none plated transformed bacteria were grown o.n as a maxi prep and purified using ZymoPURE Plasmid Maxiprep Kit (ZYMO Research).

### AAV Production-Library

HEK293T cells were seeded in 175 cm cell culture flasks to achieve 60-80% confluency before transfection. 25 μg, 250 ng or 25 ng of the AAV plasmid library, and 46 μg of pHGTI-adeno1 ¹⁸ were transfected using calcium phosphate. The molar ratio of AAV plasmid library:pHGTI-adeno1 were 1:1, 0.01:1 (30cpc), or 0.001:1 (3cpc) respectively. The 1:1 ratio was expected to receive a chimeric AAV library, in which each single particle likely contained chimeric mutation capsid proteins. The ratio of 0.01:1 and 0.001:1 was assumed to make cells receive approximately one member from the AAV plasmid library 6.and subsequently to receive a clean AAV library, in which each single particle was consisted of same mutation capsid proteins and a consistent barcode. Viral libraries were harvested and purified using iodixanol gradient as previously described ³¹. The AAV genomic titer was determined by quantification of vector DNA as described using real time PCR ³².

### Sequencing AAV Plasmid Library

To facilitate paired-end Illumina sequencing of the plasmid library, a part of the AAV plasmid was excised by Cre-recombinase to bring inserted peptide sequence and barcode closer together (FIG. 1). 1.5 μg DNA was incubated with 6U Cre-recombinase (NEB) in a volume of 100 μl at 37° C. for 1 h. The reaction was terminated at 70° C. for 10 minutes and purified by DNA Clean & Concentrator-5 (ZYMO Research). The product was digested using BsiWI and MunI, ran on agarose gel and the desired fragment was selected and purified using Zymoclean Gel DNA Recovery (Zymo Research). The gel extraction product was subjected to PreCR Repair using PreCR Repair Mix (NEB). In 50 μl reaction, 50 ng DNA, 100 μM dNTPs and 1×NAD⁺ was incubated in 1× ThermoPol Buffer at 37° C. for 20 min. 5 μl PreCR repaired DNA was PCR:ed with P5/P7 Illumina primers P11 and mix of P12, P13, P14,P15 using Phusion HSII (Thermo Fischer Scientific). Again, to reduce recombination between the fragments in PCR, an emulsion PCR was performed. The emulsion was created as previously described. The PCR cycles were 1 cycle of 30 s at 98° C., 18 cycles of 5 s at 98° C., 15 s at 63° C. and 3 min (8-fold of regular extension time) at 72° C., and 1cycle of 5 min at 72° C. The emulsion was broken, the product was purified as previously described and a PreCR Repair was performed. 5 μl PreCR repaired DNA from the previous step was used in the next emulsion PCR to add Nextera XT Indexes, using Nextera™ XT Index Kit (Illumina). The PCR program was; 1 cycle of 1 min at 98° C., 10 cycles of 15 s at 98° C., 20 s at 65° C. and 3 min (8-fold of regular extension time) at 72° C., and 1cycle of 5 min at 72° C. The product from the Nextera XT Index emulsion PCR was purified and size selected using SPRIselect Kit (Beckman Culter). The purified and indexed PCR products were sequenced using Illumina MiSeq Reagent Kit v2 (Illumina) with 150 bp paired end sequencing.

### Sequencing RNA-Derived Barcode

Total RNA was isolated from brain tissue, primary neurons and HEK293T cells using PureLink™ RNA Mini Kit (Thermo Fischer Scientific) according to the manufacture's protocol. RNA samples were incubated with DNase I (NEB) to remove DNA contamination. 5 μg RNA was incubated with 1 unit DNase 1 in 1×DNase I Reaction Buffer to a final volume of 50 μl and incubated at 37° C. for 10 minutes. Subsequently, 0.5 μl of 0.5M EDTA was added and then heat inactivated at 75° C. for 10 minutes. DNase I-treated RNA was reverse transcribed to cDNA using qScript cDNA Synthesis Kit (Quanta) according to manufacturer's recommendations. 2 μl cDNA was then amplified by PCR using primers P16 and P17. The PCR program was 1 cycle of 30 s at 98° C., 35 cycles of 5 s at 98° C., 15 s at 65° C. and 30 s at 72° C. followed by 1 cycle of 5 min at 72° C. The PCR products containing the barcodes were purified by gel extraction using Zymoclean Gel DNA Recovery Kit (Zymo Research). 20 ng purified DNA was subjected to a P5/P7 Illumina adapter PCR using primers P18 and an equal mix of P12, P13, P14, P15. The PCR cycles were 1 cycle of 30 s at 98° C., 10 cycles of 5 s at 98° C., 15 s at 65° C. and 30 s at 72° C., followed by 1 cycle of 5 min at 72° C. The PCR products were purified by gel extraction as previously described. Subsequently, a Nextera XT Index PCR using Nextera™ XT Index Kit (Illumina) was performed. The PCR program was; 1 cycle of 1 min at 98° C., 6 cycles of 15 s at 98° C., 20 s at 65° C. and 1 min at 72° C., followed by 1 cycle of 5 min at 72° C. The PCR products were purified using SPRIselect (Beckman Culter). The purified PCR products were sequenced using Illumina NextSeq™ 500/550 Mid Output Kit v2 (Illumina) with 75 bp paired end reads.

### Sequencing Viral Library

Two AAV batches were produced (for production method see “AAV production-capsid validation studies”), one batch with 100-fold dilution (30 cpc) of plasmid containing the capsids and barcode and one 1000-fold dilution (3 cpc) of the same plasmid (corresponding to 250 ng and 25 ng in “AAV production-Library”). After production, purification and titration, both batches were DNaseI treated and lyzed by Proteinase K. The viral lysate was subjected to two rounds of PCR to add Illumina compatible P5/P7 sequences and NexteraXT Indexes and then purified using SPRIselect (Beckman Culter). The purified and indexed samples were sequenced using Illumina NextSeq™ 500/550 Mid Output Kit v2 (Illumina) with 75 bp paired end reads.

### Transduction of Human ES Cells

Human ES cells were differentiated into dopaminergic progenitor cells ³⁶. 42 days after the differentiation started, cells were transduced with scAAV-GFP using 5×10⁸ gc/well and incubated o.n. 72 hours post transduction, cells were fixed with 4% PFA and stained for Map-2, Tyrosine Hydroxylase and DAPI (see Immunohistochemistry). In total 29 different AAV-capsids were validated. Cells were analysed in Cellomics, Trophos Plate runner and Confocal microscope.

### Data Assessment Workflow

A complete interaction free workflow was implemented using the R statistical package together with a number of packages from the Bioconductor repository. From these scripts, a number of broad-utility external applications (bbmap, Blast, Starcode³⁷, bowtie2, samtools, Weblogo 3 and Hammock³⁸) were called and output returned to R for further analysis. This is publically available as a Git repository at https://bitbucket.org/MNM-LU/aav-library and as a self-sustained Docker image Bjorklund/aavlib:vo.2.

In brief; barcode and sequence identification, trimming and quality filtration was conducted using the bbmap software package³⁹, which allows for kmere matching of known backbone sequences against the reads. As a vast majority of barcode reads were sequenced to the length of 20 with most barcodes of a deviating length ending up being 19 bp long, for all analysis in this study, length filtration of 18≤BC≤22 was applied.

The peptide sequence fragments were similarly isolated using the bbmap software package, but this time without any application of length restrictions and then aligned to the reference peptides using blastn.

The key component of the R-based analysis framework is a parallelized implementation of the MapReduce programming philosophy^{40, 41}. For more details on this process please refer to¹⁷. In this process Bowtie2 was first utilized to align the synthesized peptide stretches to the protein reference sequences, then blastn was used to map the sequenced fragments to the peptides and finally a purpose-built R workflow was implemented so select the pure sequencing results filtering out erroneous reads generated through template switching in the PCR based sample preparation and to identify mutations resulting from the CustonArray oligonucleotide synthesis.

From the in vitro and in vivo samples, the AAV-derived barcodes were identified by targeted sequencing and mapped back to the respective fragments and their origin within the selected proteins. Efficacy of transport was then quantified and mapped with identification of the most efficient candidates. In parallel, the barcode count together with peptide aa sequence was fed into the Hammock tool³⁸ and consensus motifs were visualized using Weblogo 3.

### Results

### Generation of a Viral Vector Library

As a first step, we identified from the literature 131 proteins with documented affinity to synapses (FIG. 1A). They fall into four major groups; viral-derived (capsid and envelope) proteins, host derived proteins (neurotrophins and disease-related proteins e.g., Tau), neurotoxins and lectins. NCBI reference sequences of the 131 proteins were translated into amino acid sequences and computationally digested into 14aa long polypeptides with a 1aa shifting sliding window approach (1. in FIG. 1B). The 61 314 peptides generated in total consisted of 44 708 unique polypeptides. Three alternative linkers were added to the polypeptides; a single Alanine (called 14aa), a rigid linker with 5 Alanine residues (14aaA5) and a flexible linker with the aa sequence GGGGS (14aaG4S) (2. in FIG. 1B). A last group of aa peptides were similarly generated with a 22aa length and a single Alanine linker (called 22aa). A total of 92 343 aa sequences were then codon optimized for expression in human cells and overhangs added to the ends to allow for directional scar-less Gibson-assembly cloning into the AAV2 Cap gene at the position N587 (3. in FIG. 1B). All resulting oligos with the total length of 170 bp were synthesized in parallel on a CustomArray oligonucleotide array.

### Generation of Highly Diverse AAV Capsid Library for the BRAVE Approach

To develop the AAV library for the BRAVE approach where peptides are displayed at N587 and peptide-associated barcodes simultaneously inserted in the AAV-genome UTR, we first generated a novel backbone plasmid. In this backbone plasmid, we combined the AAV packaging genes (Rep, Cap and AAP) under the control of the MMTV promoter and the GFP expression cassette flanked by mutated inverted terminal repeats (ITR) forming a gutted self-complementary AAV genome^{15, 16}.

To incorporate peptide sequences, we introduced an NheI site at amino acid N587 of VP1 capsid protein¹¹, in which the oligonucleotide pool was inserted. This addition of a restriction site mutates the wild-type AAV2 heparan sulfate proteoglycan binding properties¹² and this variant is hereafter referred to as AAV-MNMnull.

The resulting pool of oligonucleotides was assembled into the AAV production backbone (4. in FIG. 1B). A 20 bp random molecular barcode (BC) was simultaneously inserted in the 3′ UTR of the GFP gene using a 4-fragment Gibson-assembly reaction. Using a uni-directional Cre-recombination approach followed by emPCR-based addition of Illumina sequencing adapters, the entire pool of peptides was then sequenced using Paired-end sequencing linking the random barcodes to the respective peptide in a Look-Up table (LUT) (5a. in FIG. 1B). This approach avoids template switching during PCR and allows for near perfect matching of Barcode to inserted fragment (not shown)¹⁷. The resulting library contained 3 934 570 unique combinations of peptide and barcode containing 90 635 (and thus >98% recovery) of the designed fragments and close to 50× oversampling with barcodes. The latter is essential for noise filtration, error correction and the mapping of mutations (generated in the custom array) in the bioinformatics process below. In parallel to the LUT generation, the same plasmid library is utilized to generate replication deficient AAV viral vector preps where the peptide is displayed on the capsid surface and the barcode is packaged as part of the AAV genome (5b. in FIG. 1B). Multiple parallel screening experiments were then performed both in vitro and in vivo and after suitable selection (e.g, dissection of the targeted brain region) the mRNA was extracted and the expressed barcodes sequenced. Through the combination of the sequenced barcodes and the LUT, efficacy can be mapped back to the original 131 proteins (6 in FIG. 1B) and consensus motifs can be determined using the Hammock, hidden Markov-model (HMM) based clustering approach (7 in FIG. 1B).

### Production of AAV Library

To produce the AAV particle library, the AAV plasmid library was co-transfected together with adenoviral helper plasmid (pHGTI-adeno1) into HEK293T cells¹⁸. The AAV library plasmid was supplied at a very low concentration to ensure that the producer cell produce few (or a single) capsid variants from the AAV plasmid library^{6, 19} i.e., that each single produced virion consists only of the same mutation capsid proteins and package the correct barcode. We used 3 or 30 copies per cell (cpc) of the AAV plasmid library for production and acquired two AAV libraries with the titer of 2.5×10¹² and 4.4×10¹⁰ GC/ml for AAV-MNMlib[30cpc] and AAV-MNMlib[3cpc] respectively. To assess which peptides would allow for correct assembly and genome packaging, we DNase treated both batches (to remove non-packaged genomes), lysed the batches and sequenced the barcodes from the preps separately. Due to the very high diversity of the expressed barcodes, the overlap in barcodes was small between the productions (FIG. 1C). However, the absolute majority of all peptides packaged in the 3cpc batch were also recovered in the 30cpc batch (FIG. 1C). Injection of the AAV-MNMlib[30cpc] library into the forebrain of adult rats revealed that the inserted peptides confer a striking change in the transduction pattern compared to a AAV2-WT vector with both broader spread of transduction and retrograde transport to the connecting afferent regions (FIG. 1D). To screen for individual novel AAV capsid structures that would allow for efficient retrograde transport in neurons in vivo, we then performed a larger experiment where animals received the same library injections into the striatum from either the AAV-MNMlib[30cpc] or the AAV-MNMlib[3cpc] library prep (FIG. 1E). 8 weeks after injection, total RNA was extracted from the striatal tissue, orbitofrontal cortex (PFC), thalamus (Thal), midbrain region (SNpc) together with the injected Striatal (Str) tissue, followed by RT-PCR and massively parallel sequencing of cDNA from transcribed barcodes. Analysis of the unique peptides recovered at the different dissected regions reveal that ≈13% of the inserted peptides promote efficient transduction in vivo and ≈4% promote retrograde transport.

This example shows that libraries generated by the methods described herein can be used to identify peptides potentially conferring a desired property to a viral particle when displayed on the capsid.

### Example 2—Single-Generation BRAVE Screening In Vitro and In Vivo

### Materials and Methods

### AAV Production-Capsid Validation Studies

HEK293T cells were seeded in 175 cm cell culture flasks to achieve 60-80% confluency before transfection. 2 hours before transfection the medium was replaced with 27 ml fresh Dulbecco's modified Eagle medium (DMEM)+10% FBS+P/S. AAV was produced using standard PEI transfection ³³ using a three-plasmid system; transfer vector, modified AAV-capsid, and pHGT-1 adenoviral helper plasmid in a 1.2:1:1 ratio. PEI and plasmids were mixed in 3 ml DMEM, incubated for 15 min and then added to the cells. 16 hours post transfection 27 ml of medium was removed and equal volume of OptiPRO serum free medium (Thermo Fischer Scientific)+P/S was added. AAVs were harvested 72 hours post transfection using polyethylene glycol 8000 (PEG8000) precipitation and chloroform extraction followed by PBS exchange in Amicon Ultra-0.5 Centrifugal filters (Merck Millipore) 34. Purified AAV's were titered using qPCR with primers specific for promoter or transgene.

### In Vitro Infection

HEK293T cells were cultured in DMEM+10% FBS and P/S. Primary cortical neurons were isolated from embryonic rat (E18) or neonatal mouse of 1 day old as previously described ³⁵ and cultured in Neurobasal/B27 medium in black 96-well flat bottom culture plates (Greiner Bio One). Cells were transduced with 2×10⁶, 2×10⁷ and 2×10⁸ gc/well. AAV was added to the medium and cells were incubated overnight. AAV containing medium was replaced with fresh medium the following day. Cells were analyzed 72 hours post transduction using Cellomics (Thermo Fischer Scientific) and Plate Runner (Trophos).

### Research Animals

Adult female Sprague Dawley rats (225-250 g) used in this study were purchased from Charles River (Germany) and were housed with free access to food and water under a 12 hours light/12 hours dark cycle in a temperature-controlled room. All experimental procedures performed in this study were approved by the Ethical Committee for Use of Laboratory Animals in the Lund-Malmö region.

### Stereotaxic AAV Injection

All surgical procedures were performed under general anesthesia with a 20:1 mixture of fentanyl citrate (Fentanyl) and medetomidin hypochloride (Dormitor), injected intraperitoneally. Targeting coordinates for all stereotactic infusions were identified relative to the bregma. A small hole was drilled through the skull and the vector solutions were injected with a 25 μl Hamilton syringe fitted with a glass capillary (60-80 μm i.d. and 120-160 μm o.d.) and connected to an automatic infusion pump. Injection was carried out either unilaterally on the right side or bilaterally. The AAV library was injected unilaterally in striatum and at the following coordinates: anteroposterior, +1.2 mm; mediolateral, −2.4 mm; dorsoventral, −5.0/−4.0 mm; toothbar, −3.2 mm. Candiate AAV vectors were infused at anteroposterior, +1.2 mm; mediolateral, −2.4 mm; dorsoventral, −5.0/−4.0 mm and anteroposterior, +0.0 mm; mediolateral, −3.5 mm; dorsoventral, −5.0/−4.0 mm; toothbar, −3.2 mm. Comparative vector analysis between MNM-004-GFP and AAV-2 Retro-mCherry as well as lateralized comparison between MNM-004-GFP and MNM-004 mCherry were injected bilaterally at anteroposterior, +1.2 mm; mediolateral, −2.4/+2.4 mm; dorsoventral, −5.0/−4.0 mm. For comparative analysis of retrograde transport to midbrain dopaminergic neurons from the striatum between MNM-008, AAV-Retro and AAV-2, TH-cre animals were unilaterally injected with CTE-GFP vectors at two sites at the following coordinates: at anteroposterior, +0.8/−0.2 mm; mediolateral, −3.0/−3.7 mm; dorsoventral, −5.0/−5.0/−4.0 mm. The BLA modulation animals were injected with the MNM-004 vector at anteroposterior, +1.2 mm; mediolateral, −2.4 mm; dorsoventral, −5.0/−4.0 mm; toothbar, −3.2 mm and AAV-8 vectors at anteroposterior, −2.2 mm; mediolateral, +/−4.8 mm; dorsoventral, −7.4 mm; toothbar, −3.2 mm. For the AAV-library animals, each rat received 5 μl of vector solutions at dose of 2.5×10¹⁰ or 4.4×10⁸ vector genomes of AAV library with capsid concentration of 30cpc or 3cpc for AAV plasmid library and normal amounts pHGTI-adeno1 plasmid. The candidate vector animals received 2 μl of vector per infusions site while the animals in the BLA modulation animals were infused with 3 μl of MNM-004 in the striatum and 3 μl of Cre-inducible DREADDs AAV-8 DIO-hM3Dq/rM3Ds in the BLA. Comparative analysis between vectors injected in the striatum were injected with a viral dose of 2.3×10¹² vector genomes at a total volume of 1 μl per deposit site. All Infusions were performed at a rate of 0.2 ml/min, and the needle was left in place for an additional 3-min period before it was slowly retracted. Post-surgery, the wound was closed using surgical staples and the animal was placed on a heating mat until awake.

### Tissue Processing

Eight weeks post injection brains were processed according to subsequent post mortem analysis. For RNA extractions, animals were sacrificed using CO₂, the brains were removed and sliced in the coronal axis into two-millimeter-thick slices using a brain mold. The striatal tissue, orbitofrontal cortex, thalamus and midbrain region were rapidly dissected and frozen individually on dry ice and stored at −80° C. until RNA extraction. For immunohistochemical analysis, the animals were deeply anesthetized by sodium pentobarbital overdose (Apoteksbolaget, Sweden) and transcardially perfused with 50 ml physiological saline solution followed by 250 ml of freshly prepared, ice-cold, 4% paraformaldehyde (PFA) in 0.1 M phosphate buffer adjusted to pH=7.4. The brains were then removed and post-fixed further for 2 hours in cold PFA before storing in 25% buffered sucrose for cryoprotection over at least 24 hours until further processing. The remaining PFA fixed brains were cut into 35 mm thick coronal sections using a freezing microtome (Leica SM2000R) and collected into 8 series and stored in anti-freeze solution (0.5 M sodium phosphate buffer, 30% glycerol and 30% ethylene glycol) at −20° C.

### Immunohistochemistry

For immunohistochemical analysis, tissue sections were washed (3×) with TBS (pH 7.4) and incubated for one hour in 3% H2O2 in 0.5% TBS Triton solution in order to quench endogenous peroxidase activity and to in-crease tissue permeability. Following another washing step, the sections were blocked in 5% bovine serum and incubated for one hour and subsequently incubated with primary monoclonal antibodies overnight. To evaluate GFP expression, immunohistochemistry was performed on brain sections using primary antibody of chicken anti-GFP (1:20000; ab13970, Abcam), rM3Ds expressing neurons were identified by staining for the HA-tag (mouse anti-HA, Covance Research Products Inc Cat #MMS-101R-200 RRID: AB_10064220, 1:2000). hM3Ds expressing neurons were identified by staining for mCherry (goat anti-mCherry, LifeSpan Biosciences Cat #LS-C204207, 1:1000) Following overnight incubation, the primary antibody was washed away using TBS (x3) and then incubated with secondary antibodies for two hours. For 3, 30-di-aminobenzidine (DAB) immunohistochemistry, biotinylated anti-mouse (Vector Laboratories Cat #BA-2001 RRID: AB_2336180, 1:250), anti-goat (.Jackson ImmunoResearch Labs Cat #705-065-147 RRID: AB_2340397, 1:250) and anti-chicken (Vector Laboratories Cat #BA-9010 RRID: AB_2336114, 1:250) secondary antibodies were used. For fluorescence microscopy analysis or biotinylated goat anti chicken (1:250; BA9010, Vector laboratories) was used. For DAB immunohistochemistry, the ABC-kit (Vectorlabs) was used following incubation of the secondary antibody to amplify the staining intensity through streptavidin-peroxidase conjugation and followed by color exposure in 0.01% H₂O₂.

### Immunocytochemistry of Primary Glial and Terminally Differentiated Neurons

Analysis of vector transduction efficiency in primary glial and neurons differentiated from hESC utilized immunofluorescence detection. First the medium was removed and the cells were washed in 1x PBS. Then 100 μl of 4% PFA was added to each well containing the cells and incubated at 37 degrees for 10 minutes. Following incubation, the cells were washed with PBS. The fixed cell culture was then blocked for one hour in room temperature using 100 μl blocking solution consisting of KPBS with 5% BSA and 0.25% triton-x per well. The blocking solution was the removed and replaced with primary antibody in PBS and incubated overnight at 4 degrees. Glial cells were identified using the following antibodies: rabbit anti-GFAP (1:1000; ab7260, Abcam) and Rabbit anti-IBA-1 (1:2000; 019-19741, Wako) Following overnight incubation the wells were washed twice with KPBS and then incubated with secondary antibodies in KPBS for a total of two hours in room temperature. Secondary antibodies used included: Alexa conjugated anti-rabbit (Jackson ImmunoResearch Labs Cat #711-165-152 RRID: AB_2307443, 1:250) Finally the cells were washed twice in KPBS and left in KBPS solution for image analysis.

### Laser Scanning Confocal Microscopy

All immunofluorescence analysis was performed using the Leica SP8 microscope. Confocal images were always captured using a HyD detector with the lasers set to be activated in sequential mode, thus avoiding fluorescence signal bleed through. Solid-state lasers at wavelengths of 405, 488, 552 and 650 nm were utilized to excite the respective fluorophores. The pinhole was always retained at Airy 1 for all image acquisitions. Post acquisition, deconvolution was performed using the “Deconvolution” plugin for Image.J (developed by the Biomedical Imaging Group [BIG]-EPFL-Switzerland http://bigwww.ep.ch/) utilizing the Richardson-Lucy algorithm and applying point-spreads functions (PSFs) calculated for the specific imaging equipment using the Gibson and Lanni model in the PSF Generator (BIG, EPFL-Switzerland http://bigwww.ep.ch/algorithms/psfgenerator/).

### Results

We then utilized the BRAVE technology to screen for the re-introduction of tropism for HEK293T cells in vitro (FIG. 2B), lost when the HS binding was mutated in the AAV-MNMnull capsid (FIG. 2B′). In the screening of the 4 million uniquely barcoded capsid variants, we found several regions from the 131 included proteins that conferred a significantly improved infectivity over the parent AAV-MNMnull capsid structure (not shown). One peptide from HSV-2 surface protein pUL44 was selected and a first novel capsid was generated named AAV-MNM001 (FIG. 2A). This capsid, when used to package CMV-GFP independently, displayed a significantly increased tropism to the HEK293T cells (FIG. 2B″). In a second experiment, we used the BRAVE technology to improve the infectivity of primary cortical rat neurons in vitro. Both AAV2-WT and the AAV-MNMnull vector display very poor infectivity of primary neurons (FIG. 2D-D′) and the AAV-MNM001 displays some improvement (FIG. 2D″). Through the BRAVE screen, we identified a number of peptides clustering over a C-terminal region of the HSV-2 pUL1 protein (FIG. 2C) which when utilized alone in a novel AAV capsid (AAV-MNM002) improved the infectivity of primary neurons in culture dramatically (FIG. 2D′″). After these two proof-of-concept studies, demonstrating the validity of the single-generation BRAVE screening, we established the full scale in vivo BRAVE screening for the discovery of novel AAV capsids with improved retrograde transport capacity (not shown). From this screening, we selected 23 peptides from 19 proteins that all were represented by multiple barcodes and found in multiple animals. 23 of the 25 de novo AAV capsid structures allowed for higher than or at par with AAV2-WT packaging efficacy (FIG. 2F). Using the Hammock hidden Markov model-based clustering of all peptides with a retrograde transport ability, we could determine the putative consensus motif for each of the 23 serotypes, providing a foundation for directed optimization.

### Characterization of the AAV-MNM004 Capsid for Retrograde Transport In Vivo

In the bioinformatics analysis of the HSV pUL22 protein, a C-terminal region of the protein displayed reproducible transport to all afferent regions; Cortex, Thalamus and Substantia Nigra while not showing the same bias at the injection site in the striatum (FIG. 3A). HMM clustering of all peptides displaying these properties revealed two overlapping consensus motifs (FIG. 3C). Those were generated into the AAV-MNM004 and AAV-MNM023 capsid structures respectively with both displaying similar transport patterns in vivo (not shown) with the AAV-MNM004 capsid showing more potent transport ability and less spread at the injection site. The AAV-MNM004 capsid promoted a retrograde transport to all afferent regions as far back as the medial enthorhinal cortex while the parent AAV2-Capsid promoted efficient transduction at the site of injection but resulted in very little retrograde transport of the vector (FIG. 3D). While we were finalizing this work, another peptide was published promoting strong retrograde transport when displayed in the same location on the AAV2 capsid surface (AAV2-Retro) 9. When compared in vivo, the AAV-MNM004 capsid displayed very similar retrograde transport properties compared to AAV2 capsid with the retro peptide (Retro) injected in the contralateral striatum of the same animal (not shown). This two-fluorophore bilateral injection approach allowed us to efficiently visualize decussating versus ipsilateral projections form the PFC, the intralaminar nuclei of the thalamus and the basolateral amygdala (BLA).

This example shows that modified capsid having improved properties can be designed by identifying fragments of proteins which when displayed on the capsid confer a desired property to the modified capsid.

### Example 3—Utilization of the BRAVE Approach to Map and Understand the Function of Proteins Involved in Alzheimer's Disease, Both In Vivo and In Vitro

The BRAVE approach provides a unique possibility to systematically map protein function in vivo. We therefore utilized this approach to display peptides from endogenous proteins involved in Alzheimer's disease; APP and Tau and studied if any peptides from these proteins could promote a retrograde transport of the AAV capsid and thus provide insights into the mechanism underlying the proposed cell-to-cell communication of these proteins in the disease (FIG. 4). In the mapping of APP we found two regions that conferred retrograde transports, one in the sAAP N-terminal region and one in the Amyloid beta region (not shown). Interestingly, the sAPP region shared significant sequence homology with a region of the VP1 protein from the Theiler's murine encephalomyelitis virus (TMEV) which appears to drive its axonal uptake and infectivity (FIG. 4A). The functional properties of peptides originating from the microtubule associated protein Tau were even more striking. In this protein, a central region conveyed a very efficient retrograde transport. After deeper characterization, this region consisted of three adjacent conserved motifs with the third motif sharing significant homology with both the VSV-G glycoprotein (well used to pseudotype lenti-viruses to improve neuronal tropism) and the HIV gp120 protein (FIG. 4B-C, E). Two novel capsid structures were generated from this region, AAV-MNM009 and AAV-MNM017. Both novel capsids promoted retrograde transport in vivo but AAV-MNM017 also displayed additional interesting properties. AAV-MNM017 infected both primary neurons and primary glial cells in vitro with very high efficacy. Using this property, we then performed a displacement experiment comparing the AAV-MNM017 to the neurotrophic AAV-MNM002 capsid which is not generated from a Tau-related peptide (not shown). Three groups of primary neuron populations were pre-treated with different recombinant Tau variants (T44, T39 and K18). The T44 variant had no apparent effect on the MNM017 to MNM002 ratio of infectivity while K18 enhanced the infectivity of the MNM017 while the T39 variant efficiently blocked the infectivity compared to AAM-MNM002. This suggests that peptide derived from Tau displayed of the AAV surface is utilizing a receptor on the neurons that also has a binding activity of full length human Tau protein but that the post-translational modification of the Tau may be critical for this function. In the in vitro assessment of the 24 de novo capsid structures generated (AAV-MNM001-024) we found a subset of them (five) that displayed an improved tropism to primary glial cells in vitro (not shown). While most of the infectivity was of GFAP positive astrocytes, a few of them also infected the IBA1 positive microglia. A major hurdle for de novo capsid design using animal models has been the lack of predictability with regards to the translation to human cells. The BRAVE approach is expected to improve this through the use of naturally occurring peptides from viruses and proteins with known function in the human brain. In the human primary glia, the AAV-MNM017 retains its excellent tropism (FIG. 4D-D″).

This example shows that modified viral particles obtained by the methods disclosed herein can be used to study protein function and match functional domains.

### Example 4—Assessment of AAV Capsid Re-Shuffling Using BRAVE and Generation of Capsids Infecting DA Neurons

A number of successful studies have utilized AAV capsid reshuffling between serotypes to generate novel capsids using directed evolution. We have here utilized BRAVE to study the potential of insertion of peptides from other AAV-serotypes into the AAV2 capsid (FIG. 5). Interestingly the insertion of peptides from the same region of AAV1,2 and 8 covering the N587 aa were efficiently inserted into the AAV2 capsid. However, the same region from AAV9 did not confer the same function. Moreover, four additional regions from the N-terminal domain of VP1 (also known to be presented on the AAV capsid surface) could also be efficiently inserted. Three of these domains were conserved between AAV1,6,8 and 9 while the fourth was absent in AAV8. In a final BRAVE screening experiment, we aimed to develop a novel AAV capsid variant with efficient retrograde transport to dopamine neurons of the Substantia Nigra from injections into the Striatal output region. In this screening, we identified two regions of the CAV-2 capsid protein in close proximity (FIG. 5A-B). Interestingly, the first peptide shared significant homology with a third region of the same protein (FIG. 5A) while the second peptide (FIG. 5B) shared a peptide motif from the lectin soybean agglutinin (SA) which also efficiently transports from the synapse to the soma of neurons and can therefore be used as a retrograde tracer²⁰. The CAV-2 is an often-used viral vector for the targeting of DA neurons from the terminal in vivo²¹ and we therefore performed an experiment to confirm if this property was retained in the resulting AAV-MNM008 capsid variant. Using a Cre-inducible AAV genome (CMV-loxP-GFP) injected into the striatum of TH-Cre knock-in rats, we found that the AAV-MNM008 was more efficient in infecting the nigral neurons compared to a AAV2-WT capsid carrying the same genome (not shown). To confirm that this property was not limited to rodent DA neurons, we then performed an experiment in DA-denervated, hemi-parkinsonian, immune compromised rats. These animals first received a DA-rich neuronal transplant generated through the differentiation of Cre-expressing human embryonic stem cells (hESCs). 6 months after the transplantation of the neurons, the AAV-MNM008 vector was injected into the frontal cortex of the animals and was efficiently transported pack to the transplanted neurons innervating this region (FIG. 5C-C″). Through double fluorescence immunohistochemistry we could then confirm that the majority of these cells were indeed TH-positive and thereby inferred to be DA producing (FIG. 5C′-C″). Using the same in vitro hESC differentiation protocol we then assessed if the in vitro neuronal tropism of the de novo capsid variants would be maintained also on neurons with human origin. Indeed, all variants (AAV-MNM002, 008 and 010) which displayed high tropism on primary rodent neurons also showed much higher tropism than the wild-type AAV-variants (not shown). Of note is that the AAV-MNM004 capsid variant, so efficient in vivo was not at all suitable for in vitro transduction (not shown). Also of interest was that the AAV-MNM001, BRAVE screened in HEK293T cells of human origin was not efficient on primary rat neurons but were very efficient on human neurons (not shown), suggesting a difference in the receptor expression or structure between rodent and human cells and thus showing the value of screening directly in human cells or in humanized systems in vivo.

This example shows that the present methods can be used to improve tropism of viral particles.

### Example 5—Functional Dissection of the Basolateral Amygdala and its Involvement in the Development of Anxiety

### Materials and Methods

### Conditioned Place Preference Test

In order to assess selective DREADD activation of the BLA on conditioned place aversion, a two-chamber box was used where each chamber was separated by a wall with a closed door. Each chamber was made different from the other using visual ques on the walls and tactile sensation on the chamber floors while retaining the same light intensity. All tests were recorded using infrared illuminated CCD cameras and the animal's position recorded using the Stoelting ANY-maze 5.2 software package. On day one the animals were first adapted one of the two chambers for a total of three hours following saline injection, being alternated within the group between the two chambers to control for any chamber bias. This trial is denoted “Control”. On day two the animals were placed in the opposite chamber from day one following s.c. injection with CNO (3 mg/kg) and left in the chamber for 3 hours. This trial is labeled the “Conditioning” trial. On day three the door separating the chambers was removed and the animal was placed inside the box without any drug administration and recorded for a total of three hours for any apparent preferred chamber conditioning, denoted the “Preference test”.

### Elevated Plus Maze

To assay anxiety levels following selected activation of the BLA using DREADDs, the Elevated Plus Maze (EPM) was used. All animals paced in the EPM was recorded using Stoelting ANY-maze 5.2 software. The EPM was made out of black Plexiglas and consisted of four arms in the shape of a cross. Two of the arms (opposite of each other were open i.e., without walls while the remaining two arms were enclosed, i.e., had walls. The animals were injected with CNO (3 mg/kg) at one and a half hour prior to the start of the test. At the start of the test the animals were placed in the center of the maze and allowed to freely explore the mazes open and closed arms while being recorded for a total of 5 minutes. The animals time exploring either the open or closed arms were then quantified from the recordings in order to determine the animals' anxiety level.

### Results

We utilized the BRAVE generated AAV-MNM004 capsid variant to answer an outstanding question regarding the functional contribution of the afferents from the basolateral amygdala to the dorsal striatum (FIG. 6). This was conducted using a retrograde-induced chemogenetics (DREADD) approach. We injected the AAV-MNM004 vector expressing Cre-recombinase in the dorsal striatum and a Cre-inducible (DIO) chemogenetic (DREADD) vector into the basolateral amygdala (BLA) bilaterally into wild-type rats (FIG. 6A). After selective induction of activity of the BLA neurons projecting to the dorsal striatum using the DREADD ligand CNO, we found a striking fear and anxiety phenotype here exemplified using the elevated plus maze (EPM) where the animals spent significantly less time on the open arms (FIG. 6 B) compared to control animals (where the Cre gene was replaced with GFP). This stands in stark contrast to the believed function of the BLA projections to the ventral striatum promoting positive stimuli. This increases anxiety phenotype was accompanied by significant hypermobility in the open-field arena and a fear phenotype including excessive digging, severe sweating and episodes of freezing (FIG. 6 C). After the CNO challenges, the animals were sacrificed and the BLA stained for either the HA-tag (identifying the hM3Dq DREADD expression) or mCherry (visualizing the rM3Ds DREADD) using immunohistochemistry developed into a brown precipitation staining using the DAB-peroxidase reaction (FIG. 6 D-E).

This example shows that the modified viral vectors and particles obtainable by the methods disclosed herein can be used to help elucidate complex cellular mechanisms.

Example 6 - Sequence overview

| Sequence | | |
|---|---|---|
| ID NO: | Name | Sequence |
| 1 | AAV- | TVGPRGNASN AAPS |
| | MNM001 | |
| 2 | AAV- | ASSQSKPLAT QPPV |
| | MNM002 | |
| 3 | AAV- | NLTEYSLSRV DLGD |
| | MNM003 | |
| 4 | AAV- | YPDAVYLHRI DLGP |
| | MNM003 | |
| 5 | AAV- | VMSVLLVDTD ATQQ |
| | MNM004 | |
| 6 | AAV- | VMSVLLVDTD ATQQQLA |
| | MNM004 | |
| 7 | AAV- | VMSVLLVDTD NTQQQIA |
| | MNM004 | |
| 8 | AAV- | TDDGVSAPIL PNFH |
| | MNM005 | |
| 9 | AAV- | SALLPVGQPS HAPSVHLAAA TQ |
| | MNM006 | |
| 10 | AAV- | RTPGDEPAPA |
| | MNM007 | |
| 11 | AAV- | RTPGDEPAPA VAAQ |
| | MNM007 | |
| 12 | AAV- | FTSPLHKNEN TV |
| | MNM008 | |
| 13 | AAV- | FAYPLVKNDN HV |
| | MNM008 | |
| 14 | AAV- | SFTSPLHKNE NTVS |
| | MNM008 | |
| 15 | AAV- | FSKVSAETQA SPPE |
| | MNM009 | |
| 16 | AAV- | EDNRGINQKL AFNY |
| | MNM010 | |
| 17 | AAV- | GAYVAAN |
| | MNM011 | |
| 18 | AAV- | ADTVAAP |
| | MNM011 | |
| 19 | AAV- | TGDYVAANET HSGR |
| | MNM011 | |
| 20 | AAV- | ADSESGGHIT HSGM |
| | MNM012 | |
| 21 | AAV- | ADSESGEHIT HSGM |
| | MNM012 | |
| 22 | AAV- | ELFSSPN |
| | MNM013 | |
| 23 | AAV- | VLFSSPP |
| | MNM013 | |
| 24 | AAV- | GLIQSDQELFSSPN |
| | MNM013 | |
| 25 | AAV- | GQTGDSESVP DPQP |
| | MNM014 | |
| 26 | AAV- | GQTGDTESVP DPQP |
| | MNM014 | |
| 27 | AAV- | PAHLVNVSEG ANFT |
| | MNM015 | |
| 28 | AAV- | LVNVSEGANF T |
| | MNM015 | |
| 29 | AAV- | SALLEDPVGT VAPQI |
| | MNM016 | |
| 30 | AAV- | SALLEDPAGT VSSQI |
| | MNM016 | |
| 31 | AAV- | SIPGFPAEGS IPLP |
| | MNM017 | |
| 32 | AAV- | STLLPPELSD TTNAT |
| | MNM018 | |
| 33 | AAV- | STLLPPEVVE TANV |
| | MNM018 | |
| 34 | AAV- | EDENGTLKVT FPTP |
| | MNM019 | |
| 35 | AAV- | VDENGTPKPS SLGR |
| | MNM019 | |
| 36 | AAV- | SSTDPATGDV HAMG |
| | MNM020 | |
| 37 | AAV- | QSSSTDPATG DVHV |
| | MNM020 | |
| 38 | AAV- | QSSSTDPATG DVHA |
| | MNM020 | |
| 39 | AAV- | DPGYAETPYA SVSH |
| | MNM021 | |
| 40 | AAV- | PGGDVPPAGP GEI |
| | MNM022 | |
| 41 | AAV- | PGGEVPPAGP GAI |
| | MNM022 | |
| 42 | AAV- | LPGGEVPPAG PGAI |
| | MNM022 | |
| 43 | AAV- | QQIAAGPTEG APSV |
| | MNM023 | |
| 44 | AAV- | LVDTSGY |
| | MNM024 | |
| 45 | AAV- | YVDTSGY |
| | MNM024 | |
| 46 | AAV- | FIDISGY |
| | MNM024 | |
| 47 | AAV- | NTLVDTSGYN AEVS |
| | MNM024 | |
| 48 | AAV- | QVVAVEFDTF |
| | MNM025 | |
| 49 | AAV- | QTVAVEFDTF |
| | MNM025 | |
| 50 | AAV- | SGDQVVAVEF DTFR |
| | MNM025 | |
| 51 | AAV- | ACCGTGGGCCCCCGGGGCAACGCCAGCAACGCC |
| | MNM001 | GCCCCCAGC |
| 52 | AAV- | GCCAGCAGCCAGAGCAAGCCCCTGGCCACCCAG |
| | MNM002 | CCCCCCGTG |
| 53 | AAV- | AACCTGACCGAGTACAGCCTGAGCCGGGTGGAC |
| | MNM003 | CTGGGCGAC |
| 54 | AAV- | TACCCCGACGCCGTGTACCTGCACCGGATCGAC |
| | MNM003 | CTGGGCCCC |
| 55 | AAV- | GTGATGAGCGTGCTGCTGGTGGACACCGACGCC |
| | MNM004 | ACCCAGCAG |
| 56 | AAV- | GTGATGAGCGTGCTGCTGGTGGACACCGACGCC |
| | MNM004 | ACCCAGCAGCAGCTGGCC |
| 57 | AAV- | GTGATGAGCGTGCTGCTGGTGGACACCGACAAC |
| | MNM004 | ACCCAGCAGCAGATCGCC |
| 58 | AAV- | ACCGACGACGGCGTGAGCGCCCCCATCCTGCCC |
| | MNM005 | AACTTCCAC |
| 59 | AAV- | AGCGCCCTGCTGCCCGTGGGCCAGCCCAGCCAC |
| | MNM006 | GCCCCCAGCGTGCACCTGGCCGCCGCCACCCAG |
| 60 | AAV- | CGGACCCCCGGCGACGAGCCCGCCCCCGCC |
| | MNM007 | |
| 61 | AAV- | CGGACCCCCGGCGACGAGCCCGCCCCCGCCGTG |
| | MNM007 | GCCGCCCAG |
| 62 | AAV- | TTCACCAGCCCCCTGCACAAGAACGAGAACACC |
| | MNM008 | GTG |
| 63 | AAV- | TTCGCCTACCCCCTGGTGAAGAACGACAACCAC |
| | MNM008 | GTG |
| 64 | AAV- | AGCTTCACCAGCCCCCTGCACAAGAACGAGAAC |
| | MNM008 | ACCGTGAGC |
| 65 | AAV- | TTCAGCAAGGTGAGCGCCGAGACCCAGGCCAGC |
| | MNM009 | CCCCCCGAG |
| 66 | AAV- | GAGGACAACCGGGGCATCAACCAGAAGCTGGCC |
| | MNM010 | TTCAACTAC |
| 67 | AAV- | GGCGCCTACGTGGCCGCCAAC |
| | MNM011 | |
| 68 | AAV- | GCCGACACCGTGGCCGCCCCC |
| | MNM011 | |
| 69 | AAV- | ACCGGCGACTACGTGGCCGCCAACGAGACCCAC |
| | MNM011 | AGCGGCCGG |
| 70 | AAV- | GCCGACAGCGAGAGCGGCGGCCACATCACCCAC |
| | MNM012 | AGCGGCATG |
| 71 | AAV- | GCCGACAGCGAGAGCGGCGAGCACATCACCCAC |
| | MNM012 | AGCGGCATGGC |
| 72 | AAV- | GAGCTGTTCAGCAGCCCCAAC |
| | MNM013 | |
| 73 | AAV- | GTGCTGTTCAGCAGCCCCCCCGC |
| | MNM013 | |
| 74 | AAV- | GGCCTGATCCAGAGCGACCAGGAGCTGTTCAGC |
| | MNM013 | AGCCCCAAC |
| 75 | AAV- | GGCCAGACCGGCGACAGCGAGAGCGTGCCCGAC |
| | MNM014 | CCCCAGCCC |
| 76 | AAV- | GGCCAGACCGGCGACACCGAGAGCGTGCCCGAC |
| | MNM014 | CCCCAGCCC |
| 77 | AAV- | CCCGCCCACCTGGTGAACGTGAGCGAGGGCGCC |
| | MNM015 | AACTTCACC |
| 78 | AAV- | CTGGTGAACGTGAGCGAGGGCGCCAACTTCACC |
| | MNM015 | |
| 79 | AAV- | AGCGCCCTGCTGGAGGACCCCGTGGGCACCGTG |
| | MNM016 | GCCCCCCAG ATC |
| 80 | AAV- | AGCGCCCTGCTGGAGGACCCCGCCGGCACCGTG |
| | MNM016 | AGCAGCCAGATC |
| 81 | AAV- | AGCATCCCCGGCTTCCCCGCCGAGGGCAGCATC |
| | MNM017 | CCCCTGCCC |
| 82 | AAV- | AGCACCCTGCTGCCCCCCGAGCTGAGCGACACC |
| | MNM018 | ACCAACGCCACC |
| 83 | AAV- | AGCACCCTGCTGCCCCCCGAGGTGGTGGAGACC |
| | MNM018 | GCCAACGTG |
| 84 | AAV- | GAGGACGAGAACGGCACCCTGAAGGTGACCTTC |
| | MNM019 | CCCACCCCC |
| 85 | AAV- | GTGGACGAGAACGGCACCCCCAAGCCCAGCAGC |
| | MNM019 | CTGGGCCGG |
| 86 | AAV- | AGCAGCACCGACCCCGCCACCGGCGACGTGCAC |
| | MNM020 | GCCATGGGC |
| 87 | AAV- | CAGAGCAGCAGCACCGACCCCGCCACCGGCGAC |
| | MNM020 | GTGCACGTG |
| 88 | AAV- | CAGAGCAGCAGCACCGACCCCGCCACCGGCGAC |
| | MNM020 | GTGCACGCC |
| 89 | AAV- | GACCCCGGCTACGCCGAGACCCCCTACGCCAGC |
| | MNM021 | GTGAGCCAC |
| 90 | AAV- | CCCGGCGGCGACGTGCCCCCCGCCGGCCCCGGC |
| | MNM022 | GAGATC |
| 91 | AAV- | CCCGGCGGCGAGGTGCCCCCCGCCGGCCCCGGC |
| | MNM022 | GCCATC |
| 92 | AAV- | CTGCCCGGCGGCGAGGTGCCCCCCGCCGGCCCC |
| | MNM022 | GGCGCCATC |
| 93 | AAV- | CAGCAGATCGCCGCCGGCCCCACCGAGGGCGCC |
| | MNM023 | CCCAGCGTG |
| 94 | AAV- | CTGGTGGACACCAGCGGCTAC |
| | MNM024 | |
| 95 | AAV- | TACGTGGACACCAGCGGCTAC |
| | MNM024 | |
| 96 | AAV- | TTCATCGACATCAGCGGCTAC |
| | MNM024 | |
| 97 | AAV- | AACACCCTGGTGGACACCAGCGGCTACAACGCC |
| | MNM024 | GAGGTGAGC |
| 98 | AAV- | CAGGTGGTGGCCGTGGAGTTCGACACCTTC |
| | MNM025 | |
| 99 | AAV- | CTGACAAGACCACCCAGACCGTGGCCGTGGAGT |
| | MNM025 | TCGACACCTTCGC |
| 100 | AAV- | AGCGGCGACCAGGTGGTGGCCGTGGAGTTCGAC |
| | MNM025 | ACCTTCCGG |
| 101 | AAV2 VP1 | MAADGYLPDWLEDTLSEGIRQWWKLKPGPPPPK |
| | | PAERHKDDSRGLVLPGYKYLGPFNGLDKGEPVN |
| | | EADAAALEHDKAYDRQLDSGDNPYLKYNHADAE |
| | | FQERLKEDTSFGGNLGRAVFQAKKRVLEPLGLV |
| | | EEPVKTAPGKKRPVEHSPVEPDSSSGTGKAGQQ |
| | | PARKRLNFGQTGDADSVPDPQPLGQPPAAPSGL |
| | | GTNTMATGSGAPMADNNEGADGVGNSSGNWHCD |
| | | STWMGDRVITTSTRTWALPTYNNHLYKQISSQS |
| | | GASNDNHYFGYSTPWGYFDFNRFHCHFSPRDWQ |
| | | RLINNNWGFRPKRLNFKLFNIQVKEVTQNDGTT |
| | | TIANNLTSTVQVFTDSEYQLPYVLGSAHQGCLP |
| | | PFPADVFMVPQYGYLTLNNGSQAVGRSSFYCLE |
| | | YFPSQMLRTGNNFTFSYTFEDVPFHSSYAHSQS |
| | | LDRLMNPLIDQYLYYLSRTNTPSGTTTQSRLQF |
| | | SQAGASDIRDQSRNWLPGPCYRQQRVSKTSADN |
| | | NNSEYSWTGATKYHLNGRDSLVNPGPAMASHKD |
| | | DEEKFFPQSGVLIFGKQGSEKTNVDIEKVMITD |
| | | EEEIRTTNPVATEQYGSVSTNLQRGNRQAATAD |
| | | VNTQGVLPGMVWQDRDVYLQGPIWAKIPHTDGH |
| | | FHPSPLMGGFGLKHPPPQILIKNTPVPANPSTT |
| | | FSAAKFASFITQYSTGQVSVEIEWELQKENSKR |
| | | WNPEIQYTSNYNKSVNVDFTVDTNGVYSEPRPI |
| | | GTRYLTRNL |

### REFERENCES

1. Gray, S. J. et al. Directed evolution of a novel adeno-associated virus (AAV) vector that crosses the seizure-compromised blood-brain barrier (BBB). Mol Ther 18, 570-578 (2010).
2 Chan, K. Y. et al. Engineered AAVs for efficient noninvasive gene delivery to the central and peripheral nervous systems. Nat Neurosci 20, 1172-1179 (2017).
3. Deverman, B. E. et al. Cre-dependent selection yields AAV variants for widespread gene transfer to the adult brain. Nat Biotechnol 34, 204-209 (2016).
4. Ojala, D. S. et al. In Vivo Selection of a Computationally Designed SCHEMA AAV Library Yields a Novel Variant for Infection of Adult Neural Stem Cells in the SVZ. Mol Ther (2017).
5-Grimm, D. et al. In vitro and in vivo gene therapy vector evolution via multispecies interbreeding and retargeting of adeno-associated viruses. J Virol 82, 5887-5911 (2008).
6. Maheshri, N., Koerber, J. T., Kaspar, B. K. & Schaffer, D. V. Directed evolution of adeno-associated virus yields enhanced gene delivery vectors. Nat Biotechnol 24, 198-204 (2006).
7. Muller, O. J. et al. Random peptide libraries displayed on adeno-associated virus to select for targeted gene therapy vectors. Nat Biotechnol 21, 1040-1046 (2003).
8. Yang, L. et al. A myocardium tropic adeno-associated virus (AAV) evolved by DNA shuffling and in vivo selection. Proc Natl Acad Sci USA 106, 3946-3951 (2009).
9. Tervo, D. G. et al. A Designer AAV Variant Permits Efficient Retrograde Access to Projection Neurons. Neuron 92, 372-382 (2016).
10. Adachi, K., Enoki, T., Kawano, Y., Veraz, M. & Nakai, H. Drawing a high-resolution functional map of adeno-associated virus capsid by massively parallel sequencing. Nat Commun 5, 3075 (2014).
11. Girod, A. et al. Genetic capsid modifications allow efficient re-targeting of adeno-associated virus type 2. Nat Med 5, 1052-1056 (1999).
12. Opie, S. R., Warrington, K. H., Jr., Agbandje-McKenna, M., Zolotukhin, S. & Muzyczka, N. Identification of amino acid residues in the capsid proteins of adeno-associated virus type 2 that contribute to heparan sulfate proteoglycan binding. J Virol 77, 6995-7006 (2003).
13. Perabo, L. et al. Heparan sulfate proteoglycan binding properties of adeno-associated virus retargeting mutants and consequences for their in vivo tropism. J Virol 80, 7265-7269 (2006).
14. Ried, M. U., Girod, A., Leike, K., Buning, H. & Hallek, M. Adeno-associated virus capsids displaying immunoglobulin-binding domains permit antibody-mediated vector retargeting to specific cell surface receptors. J Virol 76, 4559-4566 (2002).
15. McCarty, D. M. et al. Adeno-associated virus terminal repeat (TR) mutant generates self-complementary vectors to overcome the rate-limiting step to transduction in vivo. Gene Ther 10, 2112-2118 (2003).
16. McCarty, D. M., Monahan, P. E. & Samulski, R. J. Self-complementary recombinant adeno-associated virus (scAAV) vectors promote efficient transduction independently of DNA synthesis. Gene Ther 8, 1248-1254 (2001).
17. Davidsson, M. et al. A novel process of viral vector barcoding and library preparation enables high-diversity library generation and recombination-free paired-end sequencing. Sci Rep 6, 37563 (2016).
18. Streck, C. J. et al. Adeno-associated virus vector-mediated systemic delivery of IFN-beta combined with low-dose cyclophosphamide affects tumor regression in murine neuroblastoma models. Clin Cancer Res 11, 6020-6029 (2005).
19. Jordan, M., Schallhorn, A. & Wurm, F. M. Transfecting mammalian cells: optimization of critical parameters affecting calcium-phosphate precipitate formation. Nucleic Acids Res 24, 596-601 (1996).
20. Shortland, P. J., Wang, H. F. & Molander, C. Transganglionic transport of the lectin soybean agglutinin (Glycine max) following injection into the sciatic nerve of the adult rat. J Neurocytol 27, 233-245 (1998).
21. Hnasko, T. S. et al. Cre recombinase-mediated restoration of nigrostriatal dopamine in dopamine-deficient mice reverses hypophagia and bradykinesia. Proc Natl Acad Sci USA 103, 8858-8863 (2006).
22. Stahl, P. L. et al. Visualization and analysis of gene expression in tissue sections by spatial transcriptomics. Science 353, 78-82 (2016).
23. Yan, Z. et al. A novel chimeric adenoassociated virus 2/human bocavirus 1 parvovirus vector efficiently transduces human airway epithelia. Mol Ther 21, 2181-2194 (2013).
24. Gray, J. T. & Zolotukhin, S. Design and construction of functional AAV vectors. Methods Mol Biol 807, 25-46 (2011).
25. Grimm, D., Kern, A., Rittner, K. & Kleinschmidt, J. A. Novel tools for production and purification of recombinant adenoassociated virus vectors. Hum Gene Ther 9, 2745-2760 (1998).
26. Ho, S. N., Hunt, H. D., Horton, R. M., Pullen, J. K. & Pease, L. R. Site-directed mutagenesis by overlap extension using the polymerase chain reaction. Gene 77, 51-59 (1989).
27. Michelfelder, S. & Trepel, M. Adeno-associated viral vectors and their redirection to cell-type specific receptors. Adv Genet 67, 29-60 (2009).
28. Chen, X., Zaro, J. L. & Shen, W. C. Fusion protein linkers: property, design and functionality. Adv Drug Deliv Rev 65, 1357-1369 (2013).
29. Reddy Chichili, V. P., Kumar, V. & Sivaraman, J. Linkers in the structural biology of protein-protein interactions. Protein Sci 22, 153-167 (2013).
30. Williams, R. et al. Amplification of complex gene libraries by emulsion PCR. Nature methods 3, 545-550 (2006).
31. Zolotukhin, S. et al. Recombinant adeno-associated virus purification using novel methods improves infectious titer and yield. Gene Ther 6, 973-985 (1999).
32 Rohr, U. P. et al. Fast and reliable titration of recombinant adeno-associated virus type-2 using quantitative real-time PCR. J Virol Methods 106, 81-88 (2002).
33. Gray, S. J. et al. Production of recombinant adeno-associated viral vectors and use in in vitro and in vivo administration. Curr Protoc Neurosci Chapter 4, Unit 4 17 (2011).
34. Wu, X., Dong, X., Wu, Z. et al A novel method for purification of recombinant adenoassociated virus vectors on a large scale. Chinese Science Bulletin 46, 485-488 (2001).
35. Brewer, G. J. & Torricelli, J. R. Isolation and culture of adult neurons and neurospheres. Nat Protoc 2, 1490-1498 (2007).
36 Nolbrant, S., Heuer, A., Parmar, M. & Kirkeby, A. Generation of high-purity human ventral midbrain dopaminergic progenitors for in vitro maturation and intracerebral transplantation. Nat Protoc 12, 1962-1979 (2017).
37. Zorita, E., Cusco, P. & Filion, G. J. Starcode: sequence clustering based on all-pairs search. Bioinformatics 31, 1913-1919 (2015).
38. Krejci, A., Hupp, T. R., Lexa, M., Vojtesek, B. & Muller, P. Hammock: a hidden Markov model-based peptide clustering algorithm to identify protein-interaction consensus motifs in large datasets. Bioinformatics 32, 9-16 (2016).
39. Bushnell, B. (Berkeley, California; 2016).
40. Dean, J. & Ghemawat, S. MapReduce: simplified data processing on large clusters. Communications of the ACM 51, 107-113 (2008).
41. Mckenna, A. et al. The Genome Analysis Toolkit: a MapReduce framework for analyzing next-generation DNA sequencing data. Genome research 20, 1297-1303 (2010).
42. Edgar, R. C. Search and clustering orders of magnitude faster than BLAST. Bioinformatics 26, 2460-2461 (2010).

### Items

1. A method of manufacturing a library of viral vectors, said method comprising:
i) selecting one or more candidate polypeptides from a group of polypeptides having or suspected of having a desired property, and retrieving the sequences of said polypeptides;
ii) providing a plurality of candidate polynucleotides, each candidate polynucleotide encoding a polypeptide fragment of one of said candidate polypeptides, such that upon transcription and translation each candidate polypeptide is represented by one or more polypeptide fragments of each candidate polypeptide;
iii) providing a plurality of barcode polynucleotides;
iv) inserting each candidate polynucleotide together with a barcode polynucleotide into a viral vector, comprising a capsid gene and a viral genome, thereby obtaining a plurality of viral vectors each comprising a single candidate polynucleotide operably linked to a barcode polynucleotide, wherein the candidate polynucleotide is inserted within the capsid gene, the capsid gene is outside the viral genome and the barcode polynucleotide is inserted within the viral genome; wherein the viral vector comprises a marker polynucleotide encoding a detectable marker;
v) amplifying the plurality of viral vectors obtained in step iv) in an amplification system, wherein each viral vector is present in a plurality of copies in the amplification system; and
a) retrieving and transferring at least a first part of the plurality of viral vectors from the amplification system of step v) in a reference system, thereby mapping each barcode polynucleotide to one candidate polynucleotide; and
b) maintaining a second part of the plurality of viral vectors in the amplification system, and optionally transferring all or part of said second part in a production system to obtain a plurality of viral particles.
2. The method of item 1, wherein the one or more polypeptide fragment is at least two overlapping polypeptide fragments.
3. The method of any one of the preceding items, wherein the viral vector is a plasmid.
4. The method of any one of the preceding items, wherein mapping each barcode polynucleotide to one candidate polynucleotide is done by sequencing a region of each viral vector of the plurality of viral vectors, said region comprising at least the barcode polynucleotide and the candidate polynucleotide.
5. The method of any one of the preceding items, wherein the sequencing is performed by next generation sequencing, such as Illumina sequencing of the region.
6. A method of designing a viral vector having a desired property, comprising the method of any one of items 1 to 5 and further comprising the steps of;
vi) retrieving a fraction of viral vectors from the amplification system of step v) b) of item 1, or retrieving at least part of the viral particles from the production system of step v) b) of item 1, and contacting a cell population with said retrieved viral vectors or viral particles;
vii) monitoring marker expression and selecting the cells wherein marker expression follows a desired pattern;
viii) identifying the barcode polynucleotides expressed in the cells selected in step vii), thereby identifying the candidate polynucleotides responsible for the desired property and the corresponding candidate polypeptides;
ix) designing a viral vector comprising a modified capsid gene, wherein the modified capsid gene comprises one of the candidate polynucleotides identified in step viii).
7. The method of item 6, further comprising the step of amplifying the viral vector obtained in step ix) in an amplification system.
8. A method of manufacturing a viral particle having a desired property, said method comprising the method of any one of items 1 to 5 and further comprising the steps of:
vi) retrieving at least part of the plurality of viral vectors from the amplification system of step v) b) or retrieving at least part of the plurality of viral particles from the production system of step v) b);
vii) contacting a cell population with the retrieved viral vectors or viral particles obtained in step vi);
viii) monitoring marker expression and selecting the cells wherein marker expression follows a desired pattern;
ix) identifying the barcode polynucleotides expressed in the cells identified in step viii), thereby identifying the candidate polynucleotides responsible for the desired property and the corresponding candidate polypeptides;
x) designing a viral vector comprising a modified capsid gene, wherein the modified capsid gene comprises one of the candidate polynucleotides identified in step ix);
xi) producing the viral vector of step x) in a production system, thereby obtaining the viral vector or the viral particle having the desired property.
9. A method of delivering a transgene to a target cell, said method comprising:
   a) providing a modified viral vector or a modified viral particle comprising a modified capsid and encapsulating a transgene, wherein the modified viral vector or the modified viral particle is the viral vector or the viral particle defined in step xi) of item 8; and
   b) injecting said modified viral vector or said modified viral particle into an injection site.
10. The method according to item 9, wherein the modified viral particle comprises a modified capsid comprising or consisting of a polypeptide which comprises or consists of a variant of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50, wherein at the most one, two or three amino acid residues have been deleted, modified or replaced.
11. A library of viral vectors, each viral vector comprising:
i) a backbone for expressing the viral vector in a host cell;
ii) a capsid gene and a candidate polynucleotide inserted therein, said candidate polynucleotide encoding a polypeptide fragment of a candidate polypeptide;
iii) a marker polynucleotide; and
iv) a barcode polynucleotide;
wherein
the candidate polypeptide is selected from a predefined group comprising one or more polypeptides having or suspected to have a desired property;
wherein upon transcription and translation each candidate polypeptide is represented by one or more polypeptide fragments in the library;
the candidate polynucleotide is inserted within the capsid gene of the viral vector so that it can be transcribed and translated to a polypeptide fragment displayed on the capsid, and is operably linked to a barcode polynucleotide inserted within the viral genome,
and the marker polynucleotide is comprised within the viral genome and the capsid gene is outside the viral genome.
12. The library of viral vectors according to any one of the preceding items, wherein the one or more polypeptide fragments is at least two overlapping polypeptide fragments.
13. The library of viral vectors according to any one of the preceding items, wherein the marker polynucleotide is the barcode polynucleotide.
14. The library of viral vectors according to any one of the preceding items, wherein the viral vector is an adeno-associated virus (AAV), a retrovirus, a lentivirus, an adeno-virus, a herpes simplex virus, a bocavirus or a rabies virus, preferably an adeno-associated virus (AAV).
15. The library of viral vectors according to any one of the preceding items, wherein the viral vector further comprises at least one replication gene.
16. The library of viral vectors according to any one of the preceding items, wherein the viral vector further comprises at least one assembly gene.
17. The library of viral vectors according to any one of the preceding items, wherein the candidate polynucleotide is located between the 5′-end of the capsid gene and the 3′-terminal-end of the capsid gene.
18. The library of viral vectors according to any one of the preceding items, wherein the marker polypeptide is selected from the group consisting of: a fluorescent protein, a bioluminescent protein, an antibiotic resistance gene, a cytotoxic gene, a surface receptor, β-galactosidase, the TVA receptor, pro-mitotic/oncogenes, trans-activators, transcription factors and Cas proteins.
19. The library of viral vectors according to any one of the preceding items, wherein the marker polynucleotide further comprises a promoter sequence.
20. The library of viral vectors according to any one of the preceding items, wherein the promoter is a constitutive promoter or an inducible promoter.
21. The library of viral vectors according to any one of the preceding items, wherein the promoter is a phosphoglycerate kinase (PGK), chicken beta actin (CBA), cytomegalovirus (CMV) early enhancer/chicken β actin (CAG), hybrid CBA (CBh), neuron-specific enolase (NSE), tyrosine hydroxylase (TH), tryptophan hydroxylase (TPH), platelet-derived growth factor (PDGF), aldehyde dehydrogenase 1 family member L1 (ALDH1L1), synapsin-1, cytomegalovirus (CMV), histone 1 (H1), U6 spliceosomal RNA (U6), calmodulin-dependent protein kinase II (CamKII), elongation factor 1-alpha (Ef1a), forkhead box J1 (Fox.J1), or glial fibrillary acidic protein (GFAP) promoter.
22. The library of viral vectors according to any one of the preceding items, wherein the barcode polynucleotide is located at the 3′ untranslated region (3′-UTR) of the marker polynucleotide.
23. The library of viral vectors according to any one of the preceding items, wherein the host cell is a mammalian cell, such as a human cell, an insect cell such as an SF9 cell or a yeast cell such as a Saccharomyces cerevisiae cell.
24. The library of viral vectors according to any one of the preceding items, wherein the host cell is a Hela cell, a primary neuron, an induced neuron, a fibroblast, an embryonic stem cell, an induced pluripotent stem cell, an insect cell such as an SF9 cell, a yeast cell or an embryonic cell, such as an embryonic kidney cell, for example HEK293 cells.
25. The library of viral vectors according to any one of the preceding items, wherein the candidate polypeptides are selected from: viral polypeptides such as a capsid polypeptide or an envelope polypeptide; polypeptides related to a disease or a disorder; polypeptides sharing a common function; neurotoxins and lectins.
26. The library of viral vectors according to any one of the preceding items, wherein the candidate polypeptides are polypeptides known to have or suspected of having a desired property.
27. The library of viral vectors according to any one of the preceding items, wherein the desired property is one or more of:
   affinity to a given cellular structure, such as a structure specific to a given type of cells, such as a synapse, or to a structure specific to a given cellular event, such as cellular division, cell differentiation, neuronal activation, inflammation or tissue damage;
   improved transport properties, such as improved transport in the environment surrounding a host cell or improved transport across the blood-brain barrier;
   increased ability to escape metabolism liver;
   increased ability to evade the immune system;
   increased ability to trigger the immune system.
28. The library of viral vectors according to any one of the preceding items, wherein the candidate polynucleotide, the marker polynucleotide and/or the barcode polynucleotide is codon-optimised.
29. A cell or a plurality of cells, comprising the library of viral vectors according to any one of items 11 to 28.
30. A viral vector encoding a viral particle for delivery of a transgene to a target cell, said viral vector comprising a modified capsid gene and a transgene to be delivered to the target cell;
wherein
the modified capsid gene is outside the viral genome and comprises or consists of a polynucleotide encoding a polypeptide improving delivery of the transgene and/or targeting to the target cell.
31. The viral vector according to item 30, wherein the viral vector is an adeno-associated virus (AAV), a retrovirus, a lentivirus, an adeno-virus, a herpes simplex virus, a bocavirus or a rabies virus.
32. The viral vector according to any one of items 30 to 31, wherein the polypeptide comprises or consists of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.
33. The viral vector according to any one of items 30 to 32, wherein the polypeptide is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.
34. The viral vector according to any one of items 30 to 33, wherein the transgene is inserted between the terminal repeat (TR) sequences of the viral genome.
35. The viral vector according to any one of items 30 to 34, wherein the viral vector upon injection to an injection site promotes retrograde transport to regions afferent to the injection site.
36. A viral particle encoded by the viral vector according to any one of items 30 to 35.
37. A modified viral vector or viral particle for delivery of a transgene to a target cell, said modified viral vector or viral particle comprising a modified capsid and a transgene to be delivered to the target cell;
wherein
the modified capsid improves one or more of: delivery of the transgene to the target cell, targeting to the target cell, infectivity of the modified viral vector or modified viral particle, and/or retrograde transport of the modified viral vector or modified viral particle compared to an unmodified viral particle comprising a native capsid gene and the transgene.
38. The modified viral vector or the modified viral particle according to item 37, wherein the modified capsid comprises or consists of a polypeptide comprising or consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49 or of SEQ ID NO: 50.
39. The modified viral vector or the modified viral particle according to any one of items 37 to 38, wherein the peptide is displayed on the modified capsid.
40. Use of a viral vector or a viral particle according to any one of items 30 to 36, or of a modified viral vector or a modified viral particle according to any one of items 37 to 40, for gene therapy.
41. A viral vector or a viral particle according to any one of items 30 to 35, or a modified viral vector or a modified viral particle according to any one of items 36 to 40, for use in a method of treatment of a disorder, such as a disorder of the nervous system.
42. The viral vector or particle or the modified viral vector or particle for the use according to item 41, wherein the disorder is selected from the group consisting of: enzyme deficiency, metabolic disorders, aggregopathy, oncogenicity, neuronal hyper- or hypo-activity, protein dysregulation and erroneous gene splicing.
43. The viral vector or particle or the modified viral vector or particle for the use according to any one of items 41 to 41, wherein the disorder is selected from the group consisting of Huntington's disease, cerebellar ataxia, multiple system atrophy, depression, epilepsy, amyotrophic lateral sclerosis, stroke, haemophilia, spinal muscular atrophy, muscular dystrophy.
44. A method for identifying a drug having a desired effect, said method comprising the steps of:
a) Providing a candidate drug;
b) Administering the candidate drug to a cell;
c) Providing a modified viral particle comprising a modified capsid allowing delivery of the viral particle to the cell of b) and a marker polynucleotide;
d) Monitoring and comparing expression and/or localisation of the marker polypeptide in the presence and absence of the candidate drug;
   thereby determining whether the candidate drug has an effect on the expression of the marker polynucleotide
   wherein the modified viral particle and/or the modified capsid are as defined in any one of the preceding items.
45. A method for improving tropism of a viral vector or particle toward a target cell, said method comprising the method of any one of items 1 to 5, and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having increased expression of the marker compared to the expression from the reference viral vector or reference viral particle;
x) designing a viral vector or a viral particle with improved tropism, comprising a modified capsid gene, wherein the modified capsid gene comprises one of the candidate polynucleotides identified in step ix).
46. A method of identifying one or more regions of a polypeptide conferring a desired property to a viral particle comprising a capsid modified by insertion of said polypeptide therein, said method comprising the method of any one of items 1 to 5, and further comprising the steps of:
vi) retrieving at least part of the plurality of viral vectors from the amplification system of step v) b) or retrieving at least part of the plurality of viral particles from the production system of step v) b);
vii) contacting a cell population comprising target cells with the retrieved viral vectors or viral particles obtained in vi) and with a reference viral vector or a reference viral particle comprising a marker;
viii) monitoring and comparing marker expression in the target cells;
ix) identifying the candidate polynucleotides in the target cells having an expression profile of the marker corresponding to the desired property, thereby identifying the region of the polypeptide responsible for said property.

1. A viral vector encoding a viral particle for delivery of a transgene to a target cell, said viral vector comprising a modified capsid gene and a transgene to be delivered to the target cell;
wherein the modified capsid gene is outside a viral genome and comprises a polynucleotide encoding a polypeptide, wherein said polypeptide improves the delivery of the transgene and/or targeting to the target cell, and
wherein said polypeptide comprises or consists of any one of SEQ ID NO: 1 to SEQ ID NO: 50.

2. The viral vector according to claim 1, wherein the target cell is a neuron, a glial cell, or a muscle myocyte.

3. The viral vector according to claim 1, wherein the viral vector is an adeno-associated virus (AAV), a retrovirus, a lentivirus, an adeno-virus, a herpes simplex virus, a bocavirus, or a rabies virus.

4. The viral vector according to claim 1, further comprising at least one replication gene and/or at least one assembly gene.

5. The viral vector according to claim 1, wherein the transgene is inserted between terminal repeat (TR) sequences of the viral genome.

6. The viral vector according to claim 1, wherein the viral vector upon injection to an injection site promotes retrograde transport to regions afferent to the injection site.

7. The viral vector according to claim 1, wherein the polypeptide is encoded by a polynucleotide comprising or consisting of any one of SEQ ID NO: 51 to 100.

8. A modified viral particle for delivery of a transgene to a target cell, said modified viral particle comprising a modified capsid and a transgene to be delivered to a host cell;
wherein the modified capsid improves one or more of: delivery of the transgene to the target cell, targeting to the target cell, infectivity of the viral particle, and/or retrograde transport of the modified viral particle compared to an unmodified viral particle comprising a native capsid gene and the transgene, and the modified capsid gene is outside the viral genome,
wherein the modified capsid comprises a polypeptide, and
wherein said polypeptide comprises or consists of any one of SEQ ID NO: 1 to SEQ ID NO: 50.

9. The modified viral particle according to claim 8, wherein the target cell is a neuron, a glial cell or a muscle myocyte.

10. The modified viral particle according to claim 8, wherein the modified viral particle is derived from an adeno-associated virus (AAV), a retrovirus, a lentivirus, an adeno-virus, a herpes simplex virus, a bocavirus or a rabies virus, optionally an adeno-associated virus (AAV).

11. The modified viral particle according to claim 8, wherein the polypeptide is encoded by a polynucleotide comprising or consisting of any one of SEQ ID NO: 51 to 100.

12. A method of treatment or prophylaxis of a disease or disorder, said method comprising the step of administering a viral vector according to claim 1 to a subject in need thereof.

13. The method according to claim 12, wherein the disorder is selected from the group consisting of: Parkinson's disease, Alzheimer's disease, schizophrenia, enzyme deficiency, metabolic disorders, aggregopathy, oncogenicity, neuronal hyper- or hypo-activity, protein dysregulation, erroneous gene splicing, Huntington's disease, cerebellar ataxia, multiple system atrophy, depression, epilepsy, amyotrophic lateral sclerosis, stroke, haemophilia, spinal muscular atrophy and muscular dystrophy.

14. A method of treatment or prophylaxis of a disease or disorder, said method comprising the step of administering a modified viral particle according to claim 8 to a subject in need thereof.

15. The method according to claim 14, wherein the disorder is Parkinson's disease, Alzheimer's disease, schizophrenia, enzyme deficiency, metabolic disorders, aggregopathy, oncogenicity, neuronal hyper- or hypo-activity, protein dysregulation, erroneous gene splicing, Huntington's disease, cerebellar ataxia, multiple system atrophy, depression, epilepsy, amyotrophic lateral sclerosis, stroke, haemophilia, spinal muscular atrophy or muscular dystrophy.

16. A method for identifying a drug having a desired effect, said method comprising the steps of:
a. providing a candidate drug;
b. administering the candidate drug to a cell;
c. providing:
i. a modified viral particle according to claim 8 comprising a modified capsid allowing delivery of the viral particle to the cell of b), and
ii. a marker polynucleotide; and
d. monitoring and comparing expression and/or localization of the marker polypeptide in the presence and absence of the candidate drug;
thereby determining whether the candidate drug has an effect on the expression of the marker polynucleotide.

17. A method of delivering a transgene to a target cell, said method comprising:
a. providing a modified viral vector according to claim 1; and
b. injecting said modified viral vector or said modified viral particle into an injection site.

18. A method of delivering a transgene to a target cell, said method comprising:
a. providing a modified viral particle according to claim 8, said modified vial particle comprising a modified capsid and encapsulating a transgene; and
b. injecting said modified viral particle into an injection site.


SEQUENCE LISTING (167 sequences)

SEQ ID NO: 1 - protein, 14 aa, herpes simplex virus type 2
TVGPRGNASNAAPS

SEQ ID NO: 2 - protein, 14 aa, herpes simplex virus type 2
ASSQSKPLATQPPV

SEQ ID NO: 3 - protein, 14 aa, herpes simplex virus type 1
NLTEYSLSRVDLGD

SEQ ID NO: 4 - protein, 14 aa, herpes simplex virus type 1
YPDAVYLHRIDLGP

SEQ ID NO: 5 - protein, 14 aa, herpes simplex virus type 1
VMSVLLVDTDATQQ

SEQ ID NO: 6 - protein, 17 aa, herpes simplex virus type 1
VMSVLLVDTDATQQQLA

SEQ ID NO: 7 - protein, 17 aa, herpes simplex virus type 1
VMSVLLVDTDNTQQQIA

SEQ ID NO: 8 - protein, 14 aa, Enterovirus 71
TDDGVSAPILPNFH

SEQ ID NO: 9 - protein, 22 aa, Glycine max
SALLPVGQPSHAPSVHLAAATQ

SEQ ID NO: 10 - protein, 10 aa, herpes B virus
RTPGDEPAPA

SEQ ID NO: 11 - protein, 14 aa, herpes B virus
RTPGDEPAPAVAAQ

SEQ ID NO: 12 - protein, 12 aa, Canine adenovirus type 2
FTSPLHKNENTV

SEQ ID NO: 13 - protein, 12 aa, Canine adenovirus type 2
FAYPLVKNDNHV

SEQ ID NO: 14 - protein, 14 aa, Canine adenovirus type 2
SFTSPLHKNENTVS

SEQ ID NO: 15 - protein, 14 aa, Homo sapiens
FSKVSAETQASPPE

SEQ ID NO: 16 - protein, 14 aa, Clostridium botulinum
EDNRGINQKLAFNY

SEQ ID NO: 17 - protein, 7 aa, Tick-borne encephalitis virus
GAYVAAN

SEQ ID NO: 18 - protein, 7 aa, Tick-borne encephalitis virus
ADTVAAP

SEQ ID NO: 19 - protein, 14 aa, Tick-borne encephalitis virus
TGDYVAANETHSGR

SEQ ID NO: 20 - protein, 14 aa, Measles virus
ADSESGGHITHSGM

SEQ ID NO: 21 - protein, 14 aa, Measles virus
ADSESGEHITHSGM

SEQ ID NO: 22 - protein, 7 aa, Armoracia rusticana
ELFSSPN

SEQ ID NO: 23 - protein, 7 aa, Armoracia rusticana
VLFSSPP

SEQ ID NO: 24 - protein, 14 aa, Armoracia rusticana
GLIQSDQELFSSPN

SEQ ID NO: 25 - protein, 14 aa, adeno-associated virus 1
GQTGDSESVPDPQP

SEQ ID NO: 26 - protein, 14 aa, adeno-associated virus 1
GQTGDTESVPDPQP

SEQ ID NO: 27 - protein, 14 aa, Pseudorabies virus
PAHLVNVSEGANFT

SEQ ID NO: 28 - protein, 11 aa, Pseudorabies virus
LVNVSEGANFT

SEQ ID NO: 29 - protein, 15 aa, herpes simplex virus type 1
SALLEDPVGTVAPQI

SEQ ID NO: 30 - protein, 15 aa, herpes simplex virus type 1
SALLEDPAGTVSSQI

SEQ ID NO: 31 - protein, 14 aa, Homo sapiens
SIPGFPAEGSIPLP

SEQ ID NO: 32 - protein, 15 aa, herpes simplex virus type 1
STLLPPELSDTTNAT

SEQ ID NO: 33 - protein, 14 aa, herpes simplex virus type 1
STLLPPEVVETANV

SEQ ID NO: 34 - protein, 14 aa, Canine adenovirus type 2
EDENGTLKVTFPTP

SEQ ID NO: 35 - protein, 14 aa, Canine adenovirus type 2
VDENGTPKPSSLGR

SEQ ID NO: 36 - protein, 14 aa, adeno-associated virus 1
SSTDPATGDVHAMG

SEQ ID NO: 37 - protein, 14 aa, adeno-associated virus 1
QSSSTDPATGDVHV

SEQ ID NO: 38 - protein, 14 aa, adeno-associated virus 1
QSSSTDPATGDVHA

SEQ ID NO: 39 - protein, 14 aa, herpes simplex virus type 2
DPGYAETPYASVSH

SEQ ID NO: 40 - protein, 13 aa, herpes simplex virus type 1
PGGDVPPAGPGEI

SEQ ID NO: 41 - protein, 13 aa, herpes simplex virus type 1
PGGEVPPAGPGAI

SEQ ID NO: 42 - protein, 14 aa, herpes simplex virus type 1
LPGGEVPPAGPGAI

SEQ ID NO: 43 - protein, 14 aa, herpes simplex virus type 1
QQIAAGPTEGAPSV

SEQ ID NO: 44 - protein, 7 aa, Clostridium botulinum
LVDTSGY

SEQ ID NO: 45 - protein, 7 aa, Clostridium botulinum
YVDTSGY

SEQ ID NO: 46 - protein, 7 aa, Clostridium botulinum
FIDISGY

SEQ ID NO: 47 - protein, 14 aa, Clostridium botulinum
NTLVDTSGYNAEVS

SEQ ID NO: 48 - protein, 10 aa, herpes simplex virus type 2
QVVAVEFDTF

SEQ ID NO: 49 - protein, 10 aa, herpes simplex virus type 2
QTVAVEFDTF

SEQ ID NO: 50 - protein, 14 aa, herpes simplex virus type 2
SGDQVVAVEFDTFR

SEQ ID NO: 51 - DNA, 42 bp, herpes simplex virus type 2
  source 1..42: mol_type = unassigned DNA
accgtgggcccccggggcaacgccagcaacgccgcccccagc

SEQ ID NO: 52 - DNA, 42 bp, herpes simplex virus type 2
  source 1..42: mol_type = unassigned DNA
gccagcagccagagcaagcccctggccacccagccccccgtg

SEQ ID NO: 53 - DNA, 42 bp, herpes simplex virus type 1
  source 1..42: mol_type = unassigned DNA
aacctgaccgagtacagcctgagccgggtggacctgggcgac

SEQ ID NO: 54 - DNA, 42 bp, herpes simplex virus type 1
  source 1..42: mol_type = unassigned DNA
taccccgacgccgtgtacctgcaccggatcgacctgggcccc

SEQ ID NO: 55 - DNA, 42 bp, herpes simplex virus type 1
  source 1..42: mol_type = unassigned DNA
gtgatgagcgtgctgctggtggacaccgacgccacccagcag

SEQ ID NO: 56 - DNA, 51 bp, herpes simplex virus type 1
  source 1..51: mol_type = unassigned DNA
gtgatgagcgtgctgctggtggacaccgacgccacccagcagcagctggcc

SEQ ID NO: 57 - DNA, 51 bp, herpes simplex virus type 1
  source 1..51: mol_type = unassigned DNA
gtgatgagcgtgctgctggtggacaccgacaacacccagcagcagatcgcc

SEQ ID NO: 58 - DNA, 42 bp, Enterovirus 71
  source 1..42: mol_type = unassigned DNA
accgacgacggcgtgagcgcccccatcctgcccaacttccac

SEQ ID NO: 59 - DNA, 66 bp, Glycine max
  source 1..66: mol_type = unassigned DNA
agcgccctgctgcccgtgggccagcccagccacgcccccagcgtgcacctggccgccgccacccag

SEQ ID NO: 60 - DNA, 30 bp, herpes B virus
  source 1..30: mol_type = unassigned DNA
cggacccccggcgacgagcccgcccccgcc

SEQ ID NO: 61 - DNA, 42 bp, herpes B virus
  source 1..42: mol_type = unassigned DNA
cggacccccggcgacgagcccgcccccgccgtggccgcccag

SEQ ID NO: 62 - DNA, 36 bp, Canine adenovirus type 2
  source 1..36: mol_type = unassigned DNA
ttcaccagccccctgcacaagaacgagaacaccgtg

SEQ ID NO: 63 - DNA, 36 bp, Canine adenovirus type 2
  source 1..36: mol_type = unassigned DNA
ttcgcctaccccctggtgaagaacgacaaccacgtg

SEQ ID NO: 64 - DNA, 42 bp, Canine adenovirus type 2
  source 1..42: mol_type = unassigned DNA
agcttcaccagccccctgcacaagaacgagaacaccgtgagc

SEQ ID NO: 65 - DNA, 42 bp, Homo sapiens
  source 1..42: mol_type = unassigned DNA
ttcagcaaggtgagcgccgagacccaggccagcccccccgag

SEQ ID NO: 66 - DNA, 42 bp, Clostridium botulinum
  source 1..42: mol_type = unassigned DNA
gaggacaaccggggcatcaaccagaagctggccttcaactac

SEQ ID NO: 67 - DNA, 21 bp, Tick-borne encephalitis virus
  source 1..21: mol_type = unassigned DNA
ggcgcctacgtggccgccaac

SEQ ID NO: 68 - DNA, 21 bp, Tick-borne encephalitis virus
  source 1..21: mol_type = unassigned DNA
gccgacaccgtggccgccccc

SEQ ID NO: 69 - DNA, 42 bp, Tick-borne encephalitis virus
  source 1..42: mol_type = unassigned DNA
accggcgactacgtggccgccaacgagacccacagcggccgg

SEQ ID NO: 70 - DNA, 42 bp, Measles virus
  source 1..42: mol_type = unassigned DNA
gccgacagcgagagcggcggccacatcacccacagcggcatg

SEQ ID NO: 71 - DNA, 44 bp, Measles virus
  source 1..44: mol_type = unassigned DNA
gccgacagcgagagcggcgagcacatcacccacagcggcatggc

SEQ ID NO: 72 - DNA, 21 bp, Armoracia rusticana
  source 1..21: mol_type = unassigned DNA
gagctgttcagcagccccaac

SEQ ID NO: 73 - DNA, 23 bp, Armoracia rusticana
  source 1..23: mol_type = unassigned DNA
gtgctgttcagcagcccccccgc

SEQ ID NO: 74 - DNA, 42 bp, Armoracia rusticana
  source 1..42: mol_type = unassigned DNA
ggcctgatccagagcgaccaggagctgttcagcagccccaac

SEQ ID NO: 75 - DNA, 42 bp, adeno-associated virus 1
  source 1..42: mol_type = unassigned DNA
ggccagaccggcgacagcgagagcgtgcccgacccccagccc

SEQ ID NO: 76 - DNA, 42 bp, adeno-associated virus 1
  source 1..42: mol_type = unassigned DNA
ggccagaccggcgacaccgagagcgtgcccgacccccagccc

SEQ ID NO: 77 - DNA, 42 bp, Pseudorabies virus
  source 1..42: mol_type = unassigned DNA
cccgcccacctggtgaacgtgagcgagggcgccaacttcacc

SEQ ID NO: 78 - DNA, 33 bp, Pseudorabies virus
  source 1..33: mol_type = unassigned DNA
ctggtgaacgtgagcgagggcgccaacttcacc

SEQ ID NO: 79 - DNA, 45 bp, herpes simplex virus type 1
  source 1..45: mol_type = unassigned DNA
agcgccctgctggaggaccccgtgggcaccgtggccccccagatc

SEQ ID NO: 80 - DNA, 45 bp, herpes simplex virus type 1
  source 1..45: mol_type = unassigned DNA
agcgccctgctggaggaccccgccggcaccgtgagcagccagatc

SEQ ID NO: 81 - DNA, 42 bp, Homo sapiens
  source 1..42: mol_type = unassigned DNA
agcatccccggcttccccgccgagggcagcatccccctgccc

SEQ ID NO: 82 - DNA, 45 bp, herpes simplex virus type 1
  source 1..45: mol_type = unassigned DNA
agcaccctgctgccccccgagctgagcgacaccaccaacgccacc

SEQ ID NO: 83 - DNA, 42 bp, herpes simplex virus type 1
  source 1..42: mol_type = unassigned DNA
agcaccctgctgccccccgaggtggtggagaccgccaacgtg

SEQ ID NO: 84 - DNA, 42 bp, Canine adenovirus type 2
  source 1..42: mol_type = unassigned DNA
gaggacgagaacggcaccctgaaggtgaccttccccaccccc

SEQ ID NO: 85 - DNA, 42 bp, Canine adenovirus type 2
  source 1..42: mol_type = unassigned DNA
gtggacgagaacggcacccccaagcccagcagcctgggccgg

SEQ ID NO: 86 - DNA, 42 bp, adeno-associated virus 1
  source 1..42: mol_type = unassigned DNA
agcagcaccgaccccgccaccggcgacgtgcacgccatgggc

SEQ ID NO: 87 - DNA, 42 bp, adeno-associated virus 1
  source 1..42: mol_type = unassigned DNA
cagagcagcagcaccgaccccgccaccggcgacgtgcacgtg

SEQ ID NO: 88 - DNA, 42 bp, adeno-associated virus 1
  source 1..42: mol_type = unassigned DNA
cagagcagcagcaccgaccccgccaccggcgacgtgcacgcc

SEQ ID NO: 89 - DNA, 42 bp, herpes simplex virus type 2
  source 1..42: mol_type = unassigned DNA
gaccccggctacgccgagaccccctacgccagcgtgagccac

SEQ ID NO: 90 - DNA, 39 bp, herpes simplex virus type 1
  source 1..39: mol_type = unassigned DNA
cccggcggcgacgtgccccccgccggccccggcgagatc

SEQ ID NO: 91 - DNA, 39 bp, herpes simplex virus type 1
  source 1..39: mol_type = unassigned DNA
cccggcggcgaggtgccccccgccggccccggcgccatc

SEQ ID NO: 92 - DNA, 42 bp, herpes simplex virus type 1
  source 1..42: mol_type = unassigned DNA
ctgcccggcggcgaggtgccccccgccggccccggcgccatc

SEQ ID NO: 93 - DNA, 42 bp, herpes simplex virus type 1
  source 1..42: mol_type = unassigned DNA
cagcagatcgccgccggccccaccgagggcgcccccagcgtg

SEQ ID NO: 94 - DNA, 21 bp, Clostridium botulinum
  source 1..21: mol_type = unassigned DNA
ctggtggacaccagcggctac

SEQ ID NO: 95 - DNA, 21 bp, Clostridium botulinum
  source 1..21: mol_type = unassigned DNA
tacgtggacaccagcggctac

SEQ ID NO: 96 - DNA, 21 bp, Clostridium botulinum
  source 1..21: mol_type = unassigned DNA
ttcatcgacatcagcggctac

SEQ ID NO: 97 - DNA, 42 bp, Clostridium botulinum
  source 1..42: mol_type = unassigned DNA
aacaccctggtggacaccagcggctacaacgccgaggtgagc

SEQ ID NO: 98 - DNA, 30 bp, herpes simplex virus type 2
  source 1..30: mol_type = unassigned DNA
caggtggtggccgtggagttcgacaccttc

SEQ ID NO: 99 - DNA, 46 bp, herpes simplex virus type 2
  source 1..46: mol_type = unassigned DNA
ctgacaagaccacccagaccgtggccgtggagttcgacaccttcgc

SEQ ID NO: 100 - DNA, 42 bp, herpes simplex virus type 2
  source 1..42: mol_type = unassigned DNA
agcggcgaccaggtggtggccgtggagttcgacaccttccgg

SEQ ID NO: 101 - protein, 735 aa, adeno-associated virus 2 [sequence omitted]

SEQ ID NO: 102 - DNA, 63 bp, synthetic construct
  misc_feature 1..63: note = Artificial/exemplary sequences
  misc_feature 1..63: note = exemplary reference sequence
  source 1..63: mol_type = other DNA
atgaagttatgggatgtcgtggctgtctgcctggtgttgctccacaccgcgtctgccttcccg

SEQ ID NO: 103 - protein, 21 aa, synthetic construct
  REGION 1..21: note = Artificial/exemplary sequences
  REGION 1..21: note = MISC_FEATURE - Exemplary reference sequence
MKLWDVVAVCLVLLHTASAFP

SEQ ID NO: 104 - protein, 14 aa, synthetic construct
  REGION 1..14: note = Artificial/exemplary sequences
  REGION 1..14: note = MISC_FEATURE - exemplary reference sequence
KLWDVVAVCLVLLH

SEQ ID NO: 105 - protein, 14 aa, synthetic construct
  REGION 1..14: note = Artificial/exemplary sequences
  REGION 1..14: note = MISC_FEATURE - exemplary reference sequence
LWDVVAVCLVLLHT

SEQ ID NO: 106 - protein, 14 aa, synthetic construct
  REGION 1..14: note = Artificial/exemplary sequences
  REGION 1..14: note = MISC_FEATURE - exemplary reference sequence
WDVVAVCLVLLHTA

SEQ ID NO: 107 - protein, 14 aa, synthetic construct
  REGION 1..14: note = Artificial/exemplary sequences
  REGION 1..14: note = MISC_FEATURE - exemplary reference sequence
DVVAVCLVLLHTAS

SEQ ID NO: 108 - protein, 14 aa, synthetic construct
  REGION 1..14: note = Artificial/exemplary sequences
  REGION 1..14: note = MISC_FEATURE - exemplary reference sequence
VVAVCLVLLHTASA

SEQ ID NO: 109 - protein, 14 aa, synthetic construct
  REGION 1..14: note = Artificial/exemplary sequences
  REGION 1..14: note = MISC_FEATURE - exemplary reference sequence
VAVCLVLLHTASAF

SEQ ID NO: 110 - protein, 24 aa, synthetic construct
  REGION 1..24: note = Artificial/exemplary sequences
  REGION 1..24: note = MISC_FEATURE - exemplary reference sequence with linkers
GGGGSVAVCLVLLHTASAFGGGGS

SEQ ID NO: 111 - DNA, 72 bp, synthetic construct
  misc_feature 1..72: note = Artificial/exemplary sequences
  misc_feature 1..72: note = exemplary reference sequence
  source 1..72: mol_type = other DNA
ggaggcggcggaagcgtggctgtctgcctggtgttgctccacaccgcgtctgccttcggaggcggcggaagc

SEQ ID NO: 112 - DNA, 20 bp, synthetic construct
  misc_feature 1..20: note = Artificial/exemplary sequences
  misc_feature 1..20: note = exemplary barcode
  source 1..20: mol_type = other DNA
aaaggctcgagtgaagatgt

SEQ ID NO: 113 - DNA, 20 bp, synthetic construct
  misc_feature 1..20: note = Artificial/exemplary sequences
  misc_feature 1..20: note = exemplary barcode
  source 1..20: mol_type = other DNA
aagcccatgtgcacgcatat

SEQ ID NO: 114 - DNA, 20 bp, synthetic construct
  misc_feature 1..20: note = Artificial/exemplary sequences
  misc_feature 1..20: note = exemplary barcode
  source 1..20: mol_type = other DNA
aaggccgggagcatagcatc

SEQ ID NO: 115 - DNA, 20 bp, synthetic construct
  misc_feature 1..20: note = Artificial/exemplary sequences
  misc_feature 1..20: note = exemplary barcode
  source 1..20: mol_type = other DNA
aaggctgggcttattggtgt

SEQ ID NO: 116 - DNA, 16 bp, synthetic construct
  misc_feature 1..16: note = Artificial/exemplary sequences
  misc_feature 1..16: note = exemplary fragment
  source 1..16: mol_type = other DNA
cggccctacgtgatgc

SEQ ID NO: 117 - DNA, 16 bp, synthetic construct
  misc_feature 1..16: note = Artificial/exemplary sequences
  misc_feature 1..16: note = exemplary fragment
  source 1..16: mol_type = other DNA
cggccctacctggccc

SEQ ID NO: 118 - DNA, 16 bp, synthetic construct
  misc_feature 1..16: note = Artificial/exemplary sequences
  misc_feature 1..16: note = exemplary fragment
  source 1..16: mol_type = other DNA
cggcccgtggtgcccc

SEQ ID NO: 119 - DNA, 17 bp, synthetic construct
  misc_feature 1..17: note = Artificial/exemplary sequences
  misc_feature 1..17: note = exemplary fragment
  source 1..17: mol_type = other DNA
cggcccgtggtgagcac

SEQ ID NO: 120 - protein, 14 aa, synthetic construct
  REGION 1..14: note = Artificial/exemplary sequences
  REGION 1..14: note = MISC_FEATURE - fragment of pUL22
VMSVLLVDTDATQQ

SEQ ID NO: 121 - protein, 14 aa, synthetic construct
  REGION 1..14: note = Artificial/exemplary sequences
  REGION 1..14: note = MISC_FEATURE - fragment of pUL22
MVDTDATQQQLAQG

SEQ ID NO: 122 - protein, 14 aa, synthetic construct
  REGION 1..14: note = Artificial/exemplary sequences
  REGION 1..14: note = MISC_FEATURE - fragment of pUL22
VDTDATQQQLAQGP

SEQ ID NO: 123 - protein, 22 aa, synthetic construct
  REGION 1..22: note = Artificial/exemplary sequences
  REGION 1..22: note = MISC_FEATURE - fragment of pUL22
PAGEVMSVLLVDTDNTQQQIAA

SEQ ID NO: 124 - protein, 14 aa, synthetic construct
  REGION 1..14: note = Artificial/exemplary sequences
  REGION 1..14: note = MISC_FEATURE - fragment of pUL22
SVLLVDTDNTQQQI

SEQ ID NO: 125 - protein, 14 aa, synthetic construct
  REGION 1..14: note = Artificial/exemplary/aligned sequence
  REGION 1..14: note = MISC_FEATURE - fragment of pUL22
VDTDNTQQQIAAGP

SEQ ID NO: 126 - protein, 14 aa, synthetic construct
  REGION 1..14: note = Artificial/exemplary sequences
  REGION 1..14: note = MISC_FEATURE - fragment of pUL22
MVDTDNTQQQIAAG

SEQ ID NO: 127 - protein, 14 aa, synthetic construct
  REGION 1..14: note = Artificial/exemplary sequences
  REGION 1..14: note = MISC_FEATURE - fragment of pUL22
EVMSVLLVDTDATQ

SEQ ID NO: 128 - protein, 22 aa, synthetic construct
  REGION 1..22: note = Artificial/exemplary sequences
  REGION 1..22: note = MISC_FEATURE - fragment of pUL22
PAGEVMSVLLVDTDATQQQLAQ

SEQ ID NO: 129 - protein, 22 aa, synthetic construct
  REGION 1..22: note = Artificial/exemplary sequences
  REGION 1..22: note = MISC_FEATURE - fragment of pUL22
EVMSVLLVDTDATQQQLAQGPV

SEQ ID NO: 130 - protein, 14 aa, synthetic construct
  REGION 1..14: note = Artificial/exemplary sequences
  REGION 1..14: note = MISC_FEATURE - fragment of pUL22
QQIAAGPTEGAPSV

SEQ ID NO: 131 - protein, 14 aa, synthetic construct
  REGION 1..14: note = Artificial/exemplary sequences
  REGION 1..14: note = MISC_FEATURE - fragment of pUL22
QIAAGPTEGAPSVF

SEQ ID NO: 132 - protein, 14 aa, synthetic construct
  REGION 1..14: note = Artificial/exemplary sequences
  REGION 1..14: note = MISC_FEATURE - fragment of pUL22
EGAPSVFSSDVPST

SEQ ID NO: 133 - protein, 14 aa, synthetic construct
  REGION 1..14: note = Artificial/exemplary sequences
  REGION 1..14: note = MISC_FEATURE - fragment of pUL22
TEGAPSVFSSDVPS

SEQ ID NO: 134 - protein, 14 aa, synthetic construct
  REGION 1..14: note = Artificial/exemplary sequences
  REGION 1..14: note = MISC_FEATURE - fragment of pUL22
QQQIAAGPTEGAPS

SEQ ID NO: 135 - protein, 22 aa, Homo sapiens
TTSEPLPQDPVKLPTTAASTPD

SEQ ID NO: 136 - protein, 14 aa, Homo sapiens
TTSEPLPQDPVKLP

SEQ ID NO: 137 - protein, 22 aa, Homo sapiens
SQSLLKTTSEPLPQDPVKLPTT

SEQ ID NO: 138 - protein, 14 aa, Homo sapiens
TSEPLPQDPVKLPT

SEQ ID NO: 139 - protein, 14 aa, Homo sapiens
QDPVKLPTTAASTP

SEQ ID NO: 140 - protein, 14 aa, Theiler's encephalomyelitis virus
DASVDFVAEPVKLP

SEQ ID NO: 141 - protein, 14 aa, Theiler's encephalomyelitis virus
SVDFVAEPVKLPEN

SEQ ID NO: 142 - protein, 14 aa, synthetic construct
  REGION 1..14: note = Artificial/exemplary sequences
  REGION 1..14: note = MISC_FEATURE - conserved motif
SIPGFPAEGSIPLP

SEQ ID NO: 143 - protein, 14 aa, synthetic construct
  REGION 1..14: note = Artificial/exemplary sequences
  REGION 1..14: note = MISC_FEATURE - conserved motif
IPGFPAEGSIPLPA

SEQ ID NO: 144 - protein, 14 aa, synthetic construct
  REGION 1..14: note = Artificial/exemplary sequences
  REGION 1..14: note = MISC_FEATURE - conserved motif
TTSIPGFPAEGSIP

SEQ ID NO: 145 - protein, 14 aa, synthetic construct
  REGION 1..14: note = Artificial/exemplary sequences
  REGION 1..14: note = MISC_FEATURE - conserved motif
SGETTSIPGFPAEG

SEQ ID NO: 146 - protein, 14 aa, synthetic construct
  REGION 1..14: note = Artificial/exemplary sequences
  REGION 1..14: note = MISC_FEATURE - conserved motif
FSKVSAETQASPPE

SEQ ID NO: 147 - protein, 14 aa, synthetic construct
  REGION 1..14: note = Artificial/exemplary sequences
  REGION 1..14: note = MISC_FEATURE - conserved motif
ADFFSKVSAETQAS

SEQ ID NO: 148 - protein, 22 aa, synthetic construct
  REGION 1..22: note = Artificial/exemplary sequences
  REGION 1..22: note = MISC_FEATURE - conserved motif
ADFFSKVSAETQASPPEGPGTG

SEQ ID NO: 149 - protein, 14 aa, synthetic construct
  REGION 1..14: note = Artificial/exemplary sequences
  REGION 1..14: note = MISC_FEATURE - conserved motif
ETQASPPEGPGTGP

SEQ ID NO: 150 - protein, 14 aa, Homo sapiens
TQASPPEGPGTGPS

SEQ ID NO: 151 - protein, 14 aa, Homo sapiens
QASPPEGPGTGPSE

SEQ ID NO: 152 - protein, 14 aa, Homo sapiens
PEGPGTGPSEEGHE

SEQ ID NO: 153 - protein, 14 aa, Homo sapiens
ETQASPPEGPGTGP

SEQ ID NO: 154 - protein, 14 aa, Vesicular stomatitis virus
APKNPGTGPAFTII

SEQ ID NO: 155 - protein, 14 aa, Vesicular stomatitis virus
SYLAPKNPGTGPVF

SEQ ID NO: 156 - protein, 14 aa, Vesicular stomatitis virus
YLAPKNPGTGPVFT

SEQ ID NO: 157 - protein, 14 aa, Vesicular stomatitis virus
LSYLAPKNPGTGPA

SEQ ID NO: 158 - protein, 14 aa, Human immunodeficiency virus
CNDKNFNGTGPCKN

SEQ ID NO: 159 - protein, 14 aa, Canine adenovirus type 2
SFTSPLHKNENTVS

SEQ ID NO: 160 - protein, 14 aa, Canine adenovirus type 2
LSFTSPLHKNENTV

SEQ ID NO: 161 - protein, 14 aa, Canine adenovirus type 2
TFAYPLVKNDNHVA

SEQ ID NO: 162 - protein, 14 aa, Canine adenovirus type 2
EDENGTLKVTFPTP

SEQ ID NO: 163 - protein, 14 aa, Canine adenovirus type 2
GLEDENGTLKVTFP

SEQ ID NO: 164 - protein, 14 aa, Glycine max
VDENGTPKPSSLGR

SEQ ID NO: 165 - protein, 14 aa, Glycine max
NKVDENGTPKPSSL

SEQ ID NO: 166 - protein, 14 aa, Glycine max
LNKVDENGTPKPSS

SEQ ID NO: 167 - protein, 14 aa, Glycine max
DENGTPKPSSLGRA